(19) United States
(12) Patent Application Publication
(10) Pub. No.: US 2026/0216460 A1
(43) Pub. Date: Jul. 30, 2026

(54) DIAGNOSTIC HEADBAND

(71) Applicant: ResMed Digital Health Inc., San Diego, CA (US)

(72) Inventors: Michael WREN, Dublin (IE); Redmond SHOULDICE, Dublin (IE); Stephen MCMAHON, Dublin (IE)

(21) Appl. No.: 19/141,019

(22) PCT Filed: Dec. 20, 2023
(86) PCT No.: PCT/IB2023/063047

§ 371 Date: Jun. 18, 2025

Related U.S. Application Data
(60) Provisional application No. 63/433,953, filed on Dec. 20, 2022.

(51) Int. Cl. A61M 16/06; A61M 16/00

(52) U.S. Cl. CPC A61M 16/0683; A61M 16/024; A61M 2205/3303; A61M 2205/3592; A61M 2209/088; A61M 2210/0618

(57) ABSTRACT

A method includes providing a head-worn assembly including an electronics module having one or more sensors. The head-worn assembly is couplable to a user interface and is wearable on a head of a user. The head-worn assembly is wearable on the head and is not coupled to the user interface when in a first configuration. The head-worn assembly is wearable on the head and is coupled to the user interface when in the second configuration. The method further includes collecting first sensor data from the one or more sensors while the user wears the head-worn assembly in the first configuration while the user is engaging in a first sleep session. The method further includes collecting second sensor data from the one or more sensors while the user wears the head-worn assembly in the second configuration while the user is wearing the user interface during a second sleep session.

### CROSS-REFERENCE TO RELATED APPLICATIONS

This application claims the benefit of, and priority to, U.S. Provisional Patent Application No. 63/433,953, filed Dec. 20, 2022, which is hereby incorporated by reference herein in its entirety.

### TECHNICAL FIELD

The present disclosure relates generally to systems and methods for monitoring sleep, and more particularly, to systems and methods for monitoring sleep before and during use of a respiratory therapy device.

### BACKGROUND

Many individuals suffer from sleep-related and/or respiratory-related disorders such as, for example, Sleep Disordered Breathing (SDB), which can include Obstructive Sleep Apnea (OSA), Central Sleep Apnea (CSA), other types of apneas such as mixed apneas and hypopneas, Respiratory Effort Related Arousal (RERA), and snoring. In some cases, these disorders manifest, or manifest more pronouncedly, when the individual is in a particular lying/sleeping position. These individuals may also suffer from other health conditions (which may be referred to as comorbidities), such as insomnia (e.g., difficulty initiating sleep, frequent or prolonged awakenings after initially falling asleep, and/or an early awakening with an inability to return to sleep), Periodic Limb Movement Disorder (PLMD), Restless Leg Syndrome (RLS), Cheyne-Stokes Respiration (CSR), respiratory insufficiency, Obesity Hyperventilation Syndrome (OHS), Chronic Obstructive Pulmonary Disease (COPD), Neuromuscular Disease (NMD), rapid eye movement (REM) behavior disorder (also referred to as RBD), dream enactment behavior (DEB), hypertension, diabetes, stroke, and chest wall disorders.

These disorders are often treated using a respiratory therapy system (e.g., a continuous positive airway pressure (CPAP) system), which delivers pressurized air to aid in preventing the individual's airway from narrowing or collapsing during sleep. However, many individuals may go undiagnosed or untreated for long periods of time, when they may otherwise have been helped by such treatment. Realizing one has a sleep-related and/or respiratory-related disorder can be difficult, especially when the immediate effects occur while the individual is asleep and when the secondary effects may be attributed to other issues. Further, even when an individual suspects they may suffer from such a disorder, that individual may be dissuaded from pursuing official diagnostic techniques, such as sleep studies.

Eventually, when a user begins respiratory therapy, there can be challenges in keeping the user on such therapy, especially if the user does not see immediate results. Some users find such systems to be uncomfortable, difficult to use, expensive, aesthetically unappealing, and/or fail to perceive the benefits associated with using the system. As a result, some users will elect not to use the respiratory therapy system or discontinue use of the respiratory therapy system if results are not seen quickly enough. The present disclosure is directed to solving these and other problems.

### SUMMARY

According to some implementations of the present disclosure, a method includes providing a head-worn assembly. The head-worn assembly includes an electronics module having one or more sensors. The head-worn assembly is couplable to a user interface and is wearable on a head of a user. The head-worn assembly is wearable on the head and is not coupled to the user interface when in a first configuration. The head-worn assembly is wearable on the head and is coupled to the user interface when in the second configuration. The method further includes collecting first sensor data from the one or more sensors while the user wears the head-worn assembly in the first configuration while the user is engaging in a first sleep session. The method further includes collecting second sensor data from the one or more sensors while the user wears the head-worn assembly in the second configuration while the user is wearing the user interface during a second sleep session.

According to some implementations of the present disclosure, a method includes providing a head-worn assembly. The head-worn assembly includes an electronics module. The electronics module includes one or more sensors. The method further including receiving first sensor data associated with a user engaging in a first sleep session while wearing the head-worn assembly and not receiving respiratory therapy. The method further including analyzing the first sensor data to generate a respiratory therapy recommendation. The method further including providing a respiratory therapy system based at least in part on the respiratory therapy recommendation. The respiratory therapy system includes a user interface. The head-worn assembly is couplable to the user interface. The method further including receiving second sensor data associated with the user engaging in a second sleep session while wearing the electronics module of the head-worn assembly coupled to the user interface.

According to some implementations of the present disclosure, a system includes a head-worn assembly. The head-worn assembly includes an electronics module. The electronics module includes one or more sensors. The head-worn assembly is wearable in a first configuration to collect first sensor data associated with a user engaging in a first sleep session without receiving respiratory therapy. The system further includes a user interface for supplying air to an airway of a user from a respiratory therapy device. When the head-worn assembly is in a second configuration, the head-worn assembly is reversibly coupled to the user interface to collect second sensor data associated with the user engaging in a second sleep session while receiving respiratory therapy via the user interface.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

FIG. 1 is a functional block diagram of a system, according to some implementations of the present disclosure;

FIG. 2 is a perspective view of at least a portion of the system of FIG. 1, a user, and a bed partner, according to some implementations of the present disclosure;

FIG. 3 is a projection view of a head-worn assembly according to certain aspects of the present disclosure;

FIG. 4A is a perspective view of a user interface, according to some implementations of the present disclosure;

FIG. 4B is an exploded view of the user interface of FIG. 4A, according to some implementations of the present disclosure;

FIG. 5 is a projection view of a head-worn assembly with a multi-part separable band according to certain aspects of the present disclosure;

FIG. 6 is a perspective view of a user interface, according to some implementations of the present disclosure;

FIG. 7 illustrates an exemplary timeline for a sleep session, according to some implementations of the present disclosure;

FIG. 8 illustrates an exemplary hypnogram associated with the sleep session of FIG. 7, according to some implementations of the present disclosure; and

FIG. 9 is a flowchart depicting a process for using a head-worn assembly according to certain aspects of the present disclosure.

FIG. 10 is a side view of a head-worn assembly base, according to certain aspects of the present disclosure.

FIG. 11 is an axonometric view of a sensor headset, according to certain aspects of the present disclosure.

FIG. 12 is a side view of a sensor headset coupled to a head-worn assembly base, according to certain aspects of the present disclosure.

FIG. 13 is a side view of a user interface coupled to a head-worn assembly base, according to certain aspects of the present disclosure.

FIG. 14 is a side view of a sensor headset and a user interface both coupled to a head-worn assembly base, according to certain aspects of the present disclosure.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

Certain aspects and features of the present disclosure relate to a head-worn assembly having an electronics module with one or more sensors. The head-worn assembly, which can take various forms (e.g., a headband, an eyepatch, an eye mask, a head covering, or the like) can be worn by a user, such as during a sleep session. Acquired sensor data can then be leveraged to provide information, such as whether or not the user would benefit from a respiratory therapy, what sort of therapy equipment would be best suited to the user, and the like. For some users, no respiratory therapy may be needed or an alternative therapy may be recommended.

For some users, respiratory therapy may be recommended. When those users start respiratory therapy, the same electronics module can be used with the respiratory therapy system, such as with a user interface of the respiratory therapy system. The electronics module may directly attach to the user interface (e.g., to a frame or headgear of a user interface), may attach to the user interface via a band, such as a band of a headband, or may otherwise couple, such as wirelessly couple, to the respiratory therapy system or an associated electronic device. The electronics module may then be able to acquire additional sensor data while the user is engaging with the respiratory therapy system. The additional sensor data can be leveraged for other purposes, such as to help ascertain an effectiveness of the respiratory therapy, to generate a recommendation to the user (e.g., to add additional pillows during the next sleep session and/or to sleep on one's side), to facilitate adjusting a parameter of the respiratory therapy system (e.g., adjusting what type of user interface is used and/or adjusting a setting of the respiratory therapy device), or the like.

The head-worn assembly can be made and provided to many users relatively easily and inexpensively, allowing many users to obtain information about their sleep. Instead of disposing of the head-worn assembly, some or all of it can be repurposed when the user begins respiratory therapy and can couple to component of the respiratory therapy system such as the user interface.

Many individuals suffer from sleep-related and/or respiratory disorders, such as Sleep Disordered Breathing (SDB) such as Obstructive Sleep Apnea (OSA), Central Sleep Apnea (CSA) and other types of apneas, Respiratory Effort Related Arousal (RERA), snoring, Cheyne-Stokes Respiration (CSR), respiratory insufficiency, Obesity Hyperventilation Syndrome (OHS), Chronic Obstructive Pulmonary Disease (COPD), Periodic Limb Movement Disorder (PLMD), Restless Leg Syndrome (RLS), Neuromuscular Disease (NMD), and chest wall disorders.

Obstructive Sleep Apnea (OSA), a form of Sleep Disordered Breathing (SDB), is characterized by events including occlusion or obstruction of the upper air passage during sleep resulting from a combination of an abnormally small upper airway and the normal loss of muscle tone in the region of the tongue, soft palate and posterior oropharyngeal wall. More generally, an apnea generally refers to the cessation of breathing caused by blockage of the air (Obstructive Sleep Apnea) or the stopping of the breathing function (often referred to as Central Sleep Apnea). CSA results when the brain temporarily stops sending signals to the muscles that control breathing. Typically, the individual will stop breathing for between about 15 seconds and about 30 seconds during an obstructive sleep apnea event.

Other types of apneas include hypopnea, hyperpnea, and hypercapnia. Hypopnea is generally characterized by slow or shallow breathing caused by a narrowed airway, as opposed to a blocked airway. Hyperpnea is generally characterized by an increase depth and/or rate of breathing. Hypercapnia is generally characterized by elevated or excessive carbon dioxide in the bloodstream, typically caused by inadequate respiration.

A Respiratory Effort Related Arousal (RERA) event is typically characterized by an increased respiratory effort for ten seconds or longer leading to arousal from sleep and which does not fulfill the criteria for an apnea or hypopnea event. RERAs are defined as a sequence of breaths characterized by increasing respiratory effort leading to an arousal from sleep, but which does not meet criteria for an apnea or hypopnea. These events fulfil the following criteria: (1) a pattern of progressively more negative esophageal pressure, terminated by a sudden change in pressure to a less negative level and an arousal, and (2) the event lasts ten seconds or longer. In some implementations, a Nasal Cannula/Pressure Transducer System is adequate and reliable in the detection of RERAs. A RERA detector may be based on a real flow signal derived from a respiratory therapy device. For example, a flow limitation measure may be determined based on a flow signal. A measure of arousal may then be derived as a function of the flow limitation measure and a measure of sudden increase in ventilation. One such method is described in WO 2008/138040 and U.S. Pat. No. 9,358,353, assigned to ResMed Ltd., the disclosure of each of which is hereby incorporated by reference herein in their entireties.

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterized by repetitive de-oxygenation and re-oxygenation of the arterial blood.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common, such as increased resistance to air movement, extended expiratory phase of respiration, and loss of the normal elasticity of the lung. COPD encompasses a group of lower airway diseases that have certain characteristics in common, such as increased resistance to air movement, extended expiratory phase of respiration, and loss of the normal elasticity of the lung.

Neuromuscular Disease (NMD) encompasses many diseases and ailments that impair the functioning of the muscles either directly via intrinsic muscle pathology, or indirectly via nerve pathology. Chest wall disorders are a group of thoracic deformities that result in inefficient coupling between the respiratory muscles and the thoracic cage.

These and other disorders are characterized by particular events (e.g., snoring, an apnea, a hypopnea, a restless leg, a sleeping disorder, choking, an increased heart rate, labored breathing, an asthma attack, an epileptic episode, a seizure, or any combination thereof) that occur when the individual is sleeping.

The Apnea-Hypopnea Index (AHI) is an index used to indicate the severity of sleep apnea during a sleep session. The AHI is calculated by dividing the number of apnea and/or hypopnea events experienced by the user during the sleep session by the total number of hours of sleep in the sleep session. The event can be, for example, a pause in breathing that lasts for at least 10 seconds. An AHI that is less than 5 is considered normal. An AHI that is greater than or equal to 5, but less than 15 is considered indicative of mild sleep apnea. An AHI that is greater than or equal to 15, but less than 30 is considered indicative of moderate sleep apnea. An AHI that is greater than or equal to 30 is considered indicative of severe sleep apnea. In children, an AHI that is greater than 1 is considered abnormal. Sleep apnea can be considered “controlled” when the AHI is normal, or when the AHI is normal or mild. The AHI can also be used in combination with oxygen desaturation levels to indicate the severity of Obstructive Sleep Apnea.

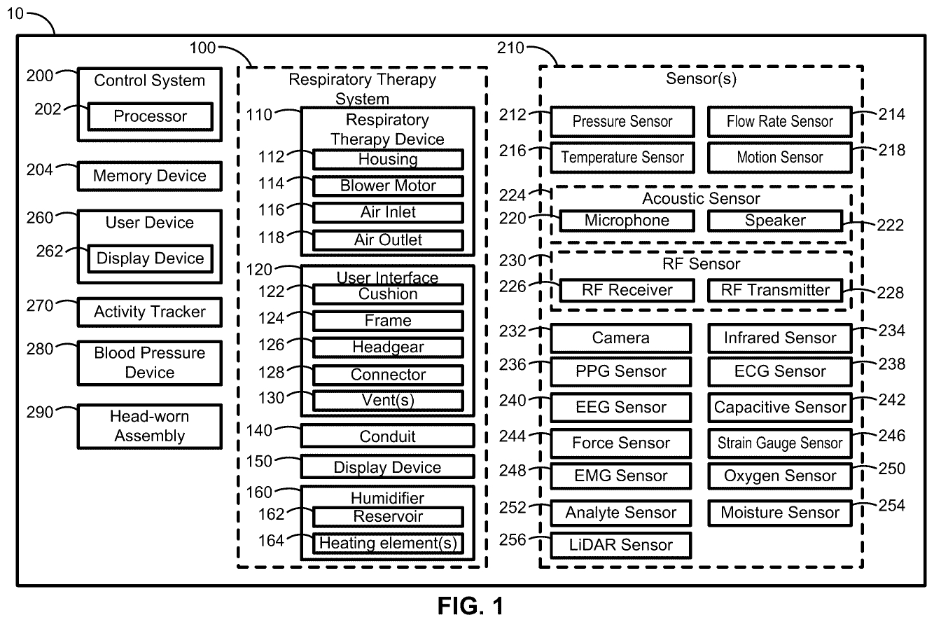

Referring to FIG. 1, a system 10, according to some implementations of the present disclosure, is illustrated. The system 10 includes a respiratory therapy system 100, a control system 200, one or more sensors 210, a user device 260, an activity tracker 270, and a head-worn assembly 290.

The respiratory therapy system 100 includes a respiratory pressure therapy (RPT) device 110 (referred to herein as respiratory therapy device 110), a user interface 120 (also referred to as a mask or a patient interface), a conduit 140 (also referred to as a tube or an air circuit), a display device 150, and a humidifier 160. Respiratory pressure therapy refers to the application of a supply of air to an entrance to a user's airways at a controlled target pressure that is nominally positive with respect to atmosphere throughout the user's breathing cycle (e.g., in contrast to negative pressure therapies such as the tank ventilator or cuirass). The respiratory therapy system 100 is generally used to treat individuals suffering from one or more sleep-related respiratory disorders (e.g., obstructive sleep apnea, central sleep apnea, or mixed sleep apnea).

The respiratory therapy system 100 can be used, for example, as a ventilator or as a positive airway pressure (PAP) system, such as a continuous positive airway pressure (CPAP) system, an automatic positive airway pressure system (APAP), a bi-level or variable positive airway pressure system (BPAP or VPAP), or any combination thereof. The CPAP system delivers a predetermined air pressure (e.g., determined by a sleep physician) to the user. The APAP system automatically varies the air pressure delivered to the user based on, for example, respiration data associated with the user. The BPAP or VPAP system is configured to deliver a first predetermined pressure (e.g., an inspiratory positive airway pressure or IPAP) and a second predetermined pressure (e.g., an expiratory positive airway pressure or EPAP) that is lower than the first predetermined pressure.

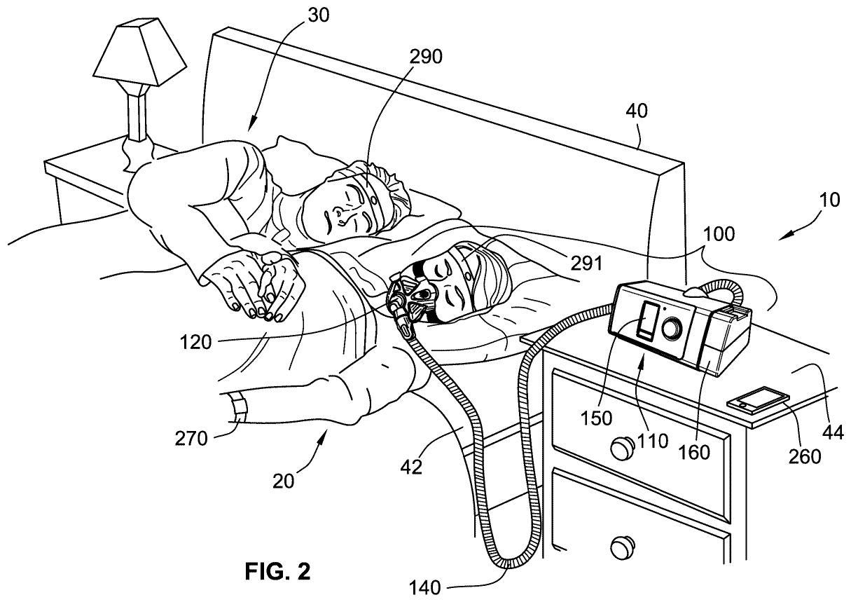

As shown in FIG. 2, the respiratory therapy system 100 can be used to treat user 20. In this example, the user 20 of the respiratory therapy system 100 and a bed partner 30 are located in a bed 40 and are laying on a mattress 42. The user interface 120 can be worn by the user 20 during a sleep session. The respiratory therapy system 100 generally aids in increasing the air pressure in the throat of the user 20 to aid in preventing the airway from closing and/or narrowing during sleep. The respiratory therapy device 110 can be positioned on a nightstand 44 that is directly adjacent to the bed 40 as shown in FIG. 2, or more generally, on any surface or structure that is generally adjacent to the bed 40 and/or the user 20.

The respiratory therapy device 110 is generally used to generate pressurized air that is delivered to a user (e.g., using one or more motors that drive one or more compressors). In some implementations, the respiratory therapy device 110 generates continuous constant air pressure that is delivered to the user. In other implementations, the respiratory therapy device 110 generates two or more predetermined pressures (e.g., a first predetermined air pressure and a second predetermined air pressure). In still other implementations, the respiratory therapy device 110 generates a variety of different air pressures within a predetermined range. For example, the respiratory therapy device 110 can deliver at least about 6 cmH₂O, at least about 10 cmH₂O, at least about 20 cmH₂O, between about 6 cmH₂O and about 10 cmH₂O, between about 7 cmH₂O and about 12 cmH₂O, etc. The respiratory therapy device 110 can also deliver pressurized air at a predetermined flow rate between, for example, about −20 L/min and about 150 L/min, while maintaining a positive pressure (relative to the ambient pressure).

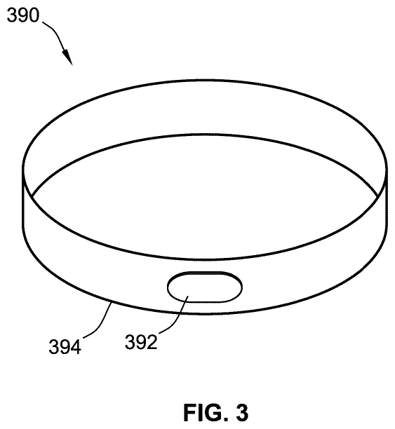

The respiratory therapy device 110 includes a housing 112, a blower motor 114, an air inlet 116, and an air outlet 118 (FIG. 1). Referring to FIGS. 3A and 3B, the blower motor 114 is at least partially disposed or integrated within the housing 112. The blower motor 114 draws air from outside the housing 112 (e.g., atmosphere) via the air inlet 116 and causes pressurized air to flow through the humidifier 160, and through the air outlet 118. In some implementations, the air inlet 116 and/or the air outlet 118 include a cover that is moveable between a closed position and an open position (e.g., to prevent or inhibit air from flowing through the air inlet 116 or the air outlet 118). As shown in FIGS. 3A and 3B, the housing 112 can include a vent 113 to allow air to pass through the housing 112 to the air inlet 116. As described below, the conduit 140 is coupled to the air outlet 118 of the respiratory therapy device 110.

Referring back to FIG. 1, the user interface 120 engages a portion of the user's face and delivers pressurized air from the respiratory therapy device 110 to the user's airway to aid in preventing the airway from narrowing and/or collapsing during sleep. This may also increase the user's oxygen intake during sleep. Generally, the user interface 120 engages the user's face such that the pressurized air is delivered to the user's airway via the user's mouth, the user's nose, or both the user's mouth and nose. Together, the respiratory therapy device 110, the user interface 120, and the conduit 140 form an air pathway fluidly coupled with an airway of the user. The pressurized air also increases the user's oxygen intake during sleep. Depending upon the therapy to be applied, the user interface 120 may form a seal, for example, with a region or portion of the user's face, to facilitate the delivery of gas at a pressure at sufficient variance with ambient pressure to effect therapy, for example, at a positive pressure of about 10 cm H₂O relative to ambient pressure. For other forms of therapy, such as the delivery of oxygen, the user interface may not include a seal sufficient to facilitate delivery to the airways of a supply of gas at a positive pressure of about 10 cmH₂O.

The user interface 120 can include, for example, a cushion 122, a frame 124, a headgear 126, connector 128, and one or more vents 130. The cushion 122 and the frame 124 define a volume of space around the mouth and/or nose of the user. When the respiratory therapy system 100 is in use, this volume space receives pressurized air (e.g., from the respiratory therapy device 110 via the conduit 140) for passage into the airway(s) of the user. The headgear 126 is generally used to aid in positioning and/or stabilizing the user interface 120 on a portion of the user (e.g., the face), and along with the cushion 122 (which, for example, can comprise silicone, plastic, foam, etc.) aids in providing a substantially air-tight seal between the user interface 120 and the user 20. In some implementations the headgear 126 includes one or more straps (e.g., including hook and loop fasteners), which may be associated with the head-worn assembly 290, as disclosed in further detail herein. The connector 128 is generally used to couple (e.g., connect and fluidly couple) the conduit 140 to the cushion 122 and/or frame 124. Alternatively, the conduit 140 can be directly coupled to the cushion 122 and/or frame 124 without the connector 128. The vent 130 can be used for permitting the escape of carbon dioxide and other gases exhaled by the user 20. The user interface 120 generally can include any suitable number of vents (e.g., one, two, five, ten, etc.).

As shown in FIG. 2, in some implementations, the user interface 120 is a facial mask (e.g., a full face mask) that covers at least a portion of the nose and mouth of the user 20. Alternatively, the user interface 120 can be a nasal mask that provides air to the nose of the user or a nasal pillow mask that delivers air directly to the nostrils of the user 20. In other implementations, the user interface 120 includes a mouthpiece (e.g., a night guard mouthpiece molded to conform to the teeth of the user, a mandibular repositioning device, etc.).

In some cases, a user 20 or bed partner 30 may make use of a head-worn assembly 291, 290. The head-worn assembly 290 used by bed partner 30 shows a head-worn assembly 290 being used without the bed partner 30 engaging in respiratory therapy. For example, the bed partner 30 may be using the head-worn assembly 290 to monitor their sleep to determine whether or not respiratory therapy would be beneficial. User 20 may be making use of head-worn assembly 291 while the user is making use of a respiratory therapy system 100. User 20, prior to using the respiratory therapy system 100, may have used the head-worn assembly 291 to determine that respiratory therapy would be beneficial to the user 20, which may have prompted the user 20 in obtaining the respiratory therapy system 100. Now that the user is making use of the respiratory therapy system 100, the head-worn assembly 291 may be physically coupled to the respiratory therapy system 100, such as via the user interface 120; may be electrically coupled to the respiratory therapy system 100, such as via an electrical connection passing through conduit 140; and/or may be communicatively coupled to the respiratory therapy system 100 and/or user device 260, such as via a wired or wireless connection. Sensor data from the head-worn assembly 291 may be used to monitor the sleep of the user 20, make recommendations for the user 20, and/or facilitate making changes to the user's respiratory therapy (e.g., altering a parameter of the respiratory therapy system 100).

FIG. 3 is a projection view of a head-worn assembly 390 according to certain aspects of the present disclosure. Head-worn assembly 390 can be any suitable head-worn assembly, such as head-worn assembly 190 of FIG. 1, head-worn assembly 290 of FIG. 2, or head-worn assembly 291 of FIG. 2.

The head-worn assembly 390 depicted in FIG. 3 takes the form of a headband. The head-worn assembly 390 includes an electronics module 392 coupled to a band 394. The electronics module 392 can be removably coupled to the band 394 in any suitable fashion, such as being place within a pocket of the band 394 or being snapped or otherwise secured to the band 394 by fasteners, snaps, or other attachment hardware. In some cases, electronics module 392 is permanently coupled to band 394. Band 394 can be made of or include an elastic material, or can be otherwise formed to provide sufficient elasticity to comfortably fit on the head of a user. In some cases, band 394 includes or is made of an elastic, woven material.

Electronics module 392 can contain one or more sensors. Any suitable sensors can be used, such as one or more of the one or more sensors 210 of FIG. 1. In some cases, electronics module 392 includes at least i) one or more motion sensors; ii) one or more acoustic sensors; iii) one or more PPG sensors; iv) one or more temperatures sensors; or v) any combination of i-iv.

While head-worn assembly 390 is depicted as a headband, in some cases it may take other forms, such as an eye patch, an eye mask, a head covering (e.g., a hat, cap, hood, or the like), or other forms. In some cases, the head-worn assembly can include or otherwise take the form of i) a headband; ii) an eyepatch; iii) an eye mask; iv) a head covering; v) a nasal strip; vi) an ear bud or ear buds; vii) an audio headset (e.g., headphones); viii) a hearing aid or hearing aids; ix) a pair of glasses; x) a scarf; xi) a head-worn video display (e.g., an augmented reality headset or virtual reality headset); or xii) any combination of i-xi.

Additionally, while the band 394 is depicted as being a continuous piece of material, in some cases band 394 can be made of multiple pieces of materials coupled together, or a single piece of material that couples together to form a loop (e.g., via a buckle or hook-and-loop fastener).

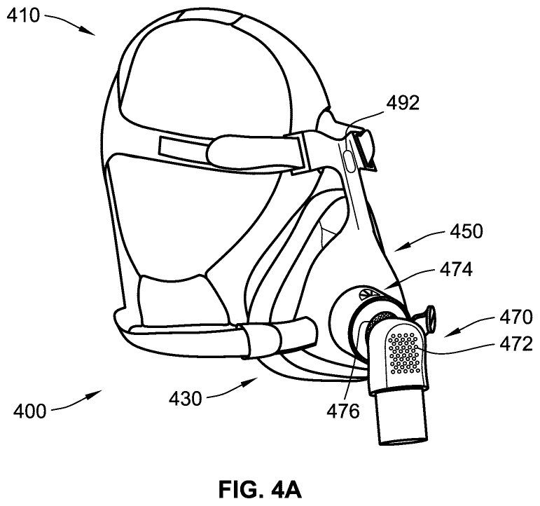

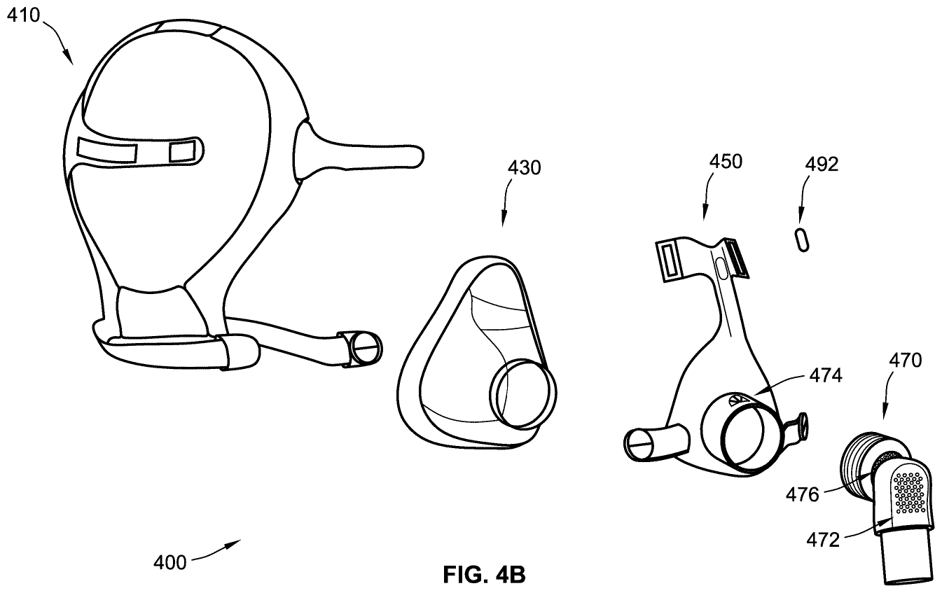

While not visible in FIG. 3, in some cases, head-worn assembly 390 includes additional bands or extensions from band 394 that facilitate coupling the head-worn assembly 390 to a user interface, such as via band 394. In some other cases, the electronics module 392 of the head-worn assembly 390 is removed from a remainder of the head-worn assembly 390 (e.g., removed from band 394) before being coupled to a user interface, such as depicted in FIGS. 4A and 4B.

Referring to FIGS. 4A and 4B, a user interface 400 that is the same as, or similar to, the user interface 120 (FIG. 1) according to some implementations of the present disclosure is illustrated. The user interface 400 generally includes a cushion 430 and a frame 450 that define a volume of space around the mouth and/or nose of the user. When in use, the volume of space receives pressurized air for passage into the user's airways. In some implementations, the cushion 430 and frame 450 of the user interface 400 form a unitary component of the user interface. The user interface 400 can also include a headgear 410, which generally includes a strap assembly and optionally a connector 470. The headgear 410 is configured to be positioned generally about at least a portion of a user's head when the user wears the user interface 400. The headgear 410 can be coupled to the frame 450 and positioned on the user's head such that the user's head is positioned between the headgear 410 and the frame 450. The cushion 430 is positioned between the user's face and the frame 450 to form a seal on the user's face. The optional connector 470 is configured to couple to the frame 450 and/or cushion 430 at one end and to a conduit of a respiratory therapy device (not shown). The pressurized air can flow directly from the conduit of the respiratory therapy system into the volume of space defined by the cushion 430 (or cushion 430 and frame 450) of the user interface 400 through the connector 470). From the user interface 400, the pressurized air reaches the user's airway through the user's mouth, nose, or both. Alternatively, where the user interface 400 does not include the connector 470, the conduit of the respiratory therapy system can connect directly to the cushion 430 and/or the frame 450.

In some implementations, the connector 470 may include one or more vents 472 (e.g., a plurality of vents) located on the main body of the connector 470 itself and/or one or a plurality of vents 476 (“diffuser vents”) in proximity to the frame 450, for permitting the escape of carbon dioxide (CO₂) and other gases exhaled by the user. In some implementations, one or a plurality of vents, such as vents 472 and/or 476 may be located in the user interface 400, such as in frame 450, and/or in the conduit 140. In some implementations, the frame 450 includes at least one anti-asphyxia valve (AAV) 474, which allows CO₂ and other gases exhaled by the user to escape in the event that the vents (e.g., the vents 472 or 476) fail when the respiratory therapy device is active. In general, AAVs (e.g., the AAV 474) are present for full face masks (e.g., as a safety feature); however, the diffuser vents and vents located on the mask or connector (usually an array of orifices in the mask material itself or a mesh made of some sort of fabric, in many cases replaceable) are not necessarily both present (e.g., some masks might have only the diffuser vents such as the plurality of vents 476, other masks might have only the plurality of vents 472 on the connector itself).

In some cases, a portion of the user interface 400 can be configured to receive a head-worn assembly, such as an electronics module of a head-worn assembly. As depicted in FIGS. 4A and 4B, the frame 450 includes a receiving location for receiving an electronics module 492 (e.g., electronics module 392 after removal from the remainder of the head-worn assembly 390 of FIG. 3). The receiving location can include one or more features to mechanically, electrically, and/or communicatively couple to the electronics module 492, such as magnets, snaps, a pocket, or the like. Thus, once the head-worn assembly (e.g., the electronics module 492 after removal of the remainder of the head-worn assembly) is coupled to the user interface 400, its sensors can be used to monitor the user while the user is making use of the respiratory therapy system.

While a particular style of user interface 400 is depicted, other styles of user interface can be used.

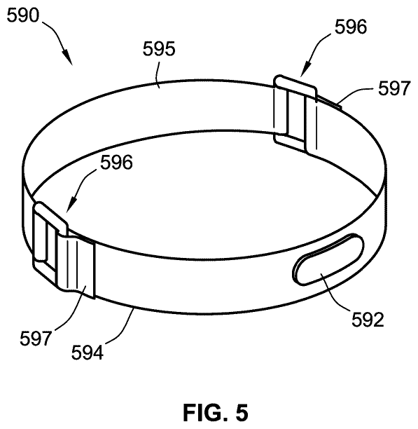

FIG. 5 is a projection view of a head-worn assembly 590 with a multi-part separable band 594 according to certain aspects of the present disclosure. Head-worn assembly 590 can be similar to head-worn assembly 390 of FIG. 3. Electronics module 592 and band 594 can be similar to electronics module 392 and band 394 of FIG. 3, except with band 594 being a multi-part band.

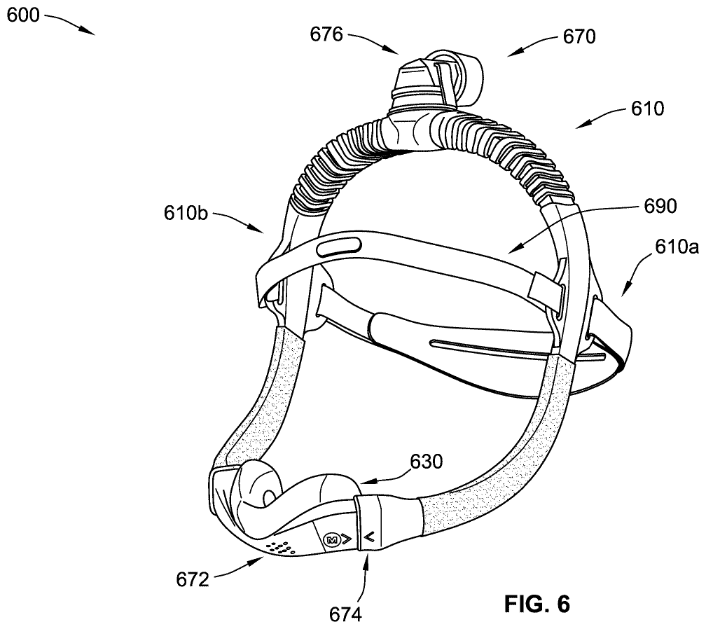

The multi-part band 594 can include a front portion 598 coupled to a rear portion 595. The rear portion 595 can have two ends 596, each of which having fittings or openings for receiving corresponding ends 597 of the front portion 598. The corresponding ends 597 of the front portion, after passing through the fittings or openings of ends 596, can attach to the remainder of the front portion 598, such as via a hook-and-loop fastener. Thus, ends 597 can be easily adjusted to ensure the band 594 fits well on the user's head, and to permit the front portion 598 to be removed from the rear portion 595, such as to allow the front portion 598 to couple to a respiratory therapy system (e.g., to headgear of a user interface of a respiratory therapy system), such as depicted in FIG. 6.

Referring to FIG. 6, a user interface 600 that is the same as, or similar to, the user interface 120 (FIG. 1) according to some implementations of the present disclosure is illustrated. The user interface 600 is similar to the user interface 500 in that it is an indirect user interface. The indirect headgear user interface 600 includes headgear 610, a cushion 630, and a connector 670. The headgear 610 includes strap 610a and a headgear conduit 610b. Similar to the user interface 400 (FIGS. 4A-4B) and user interface 500 (FIGS. 5A-5B), the headgear 610 is configured to be positioned generally about at least a portion of a user's head when the user wears the user interface 600. The headgear 610 includes a strap 610a that can be coupled to the headgear conduit 610b and positioned on the user's head such that the user's head is positioned between the strap 610a and the headgear conduit 610b. The cushion 630 is positioned between the user's face and the headgear conduit 610b to form a seal on the user's face.

The connector 670 is configured to couple to the headgear 610 at one end and a conduit of the respiratory therapy system at the other end (e.g., conduit 140). In other implementations, the connector 670 is not included and the headgear 610 can alternatively connect directly to conduit of the respiratory therapy system. The headgear conduit 610b can be configured to deliver pressurized air from the conduit of the respiratory therapy system to the cushion 630, or more specifically, to the volume of space around the mouth and/or nose of the user and enclosed by the user cushion. The headgear conduit 610b is hollow to provide a passageway for the pressurized air. Both sides of the headgear conduit 610b can be hollow to provide two passageways for the pressurized air. Alternatively, only one side of the headgear conduit 610b can be hollow to provide a single passageway. In the implementation illustrated in FIGS. 6A and 6B, headgear conduit 610b comprises two passageways which, in use, are positioned at either side of a user's head/face. Alternatively, only one passageway of the headgear conduit 610b can be hollow to provide a single passageway. The pressurized air can flow from the conduit of the respiratory therapy system, through the connector 670 and the headgear conduit 610b, and into the volume of space between the cushion 630 and the user's face. From the volume of space between the cushion 630 and the user's face, the pressurized air reaches the user's airway through the user's mouth, nose, or both.

In some implementations, the cushion 630 includes a plurality of vents 672 on the cushion 630 itself. Additionally or alternatively, in some implementations, the connector 670 includes a plurality of vents 676 (“diffuser vents”) in proximity to the headgear 610, for permitting the escape of carbon dioxide (CO₂) and other gases exhaled by the user when the respiratory therapy device is active. In some implementations, the headgear 610 may include at least one plus anti-asphyxia valve (AAV) 674 in proximity to the cushion 630, which allows CO₂ and other gases exhaled by the user to escape in the event that the vents (e.g., the vents 672 or 676) fail when the respiratory therapy device is active.

In some cases, a head-worn assembly 690 can be coupled to a user interface 100. As depicted in FIG. 6, the front portion of a band of a head-worn assembly 690 is removably coupled to user interface 100 by passing through corresponding openings on headgear 610. The ends of the front portion of the band of the head-worn assembly 690 can be coupled to the remainder of the band, such as via a hook-and-loop fastener, permitting adjustment of the head-worn assembly 690 while it is coupled to user interface 100.

Referring back to FIG. 1, the conduit 140 (also referred to as an air circuit or tube) allows the flow of air between components of the respiratory therapy system 100, such as between the respiratory therapy device 110 and the user interface 120. In some implementations, there can be separate limbs of the conduit for inhalation and exhalation. In other implementations, a single limb conduit is used for both inhalation and exhalation.

In an example, the conduit 140 includes a first end that is coupled to the air outlet 118 of the respiratory therapy device 110. The first end can be coupled to the air outlet 118 of the respiratory therapy device 110 using a variety of techniques (e.g., a press fit connection, a snap fit connection, a threaded connection, etc.). In some implementations, the conduit 140 includes one or more heating elements that heat the pressurized air flowing through the conduit 140 (e.g., heat the air to a predetermined temperature or within a range of predetermined temperatures). Such heating elements can be coupled to and/or imbedded in the conduit 140. In such implementations, the first end can include an electrical contact that is electrically coupled to the respiratory therapy device 110 to power the one or more heating elements of the conduit 140. For example, the electrical contact can be electrically coupled to an electrical contact of the air outlet 118 of the respiratory therapy device 110. In this example, electrical contact of the conduit 140 can be a male connector and the electrical contact of the air outlet 118 can be female connector, or, alternatively, the opposite configuration can be used.

The display device 150 is generally used to display image(s) including still images, video images, or both and/or information regarding the respiratory therapy device 110. For example, the display device 150 can provide information regarding the status of the respiratory therapy device 110 (e.g., whether the respiratory therapy device 110 is on/off, the pressure of the air being delivered by the respiratory therapy device 110, the temperature of the air being delivered by the respiratory therapy device 110, etc.) and/or other information (e.g., a sleep score and/or a therapy score, also referred to as a myAir™ score, such as described in WO 2016/061629 and U.S. Patent Pub. No. 2017/0311879, which are hereby incorporated by reference herein in their entireties, the current date/time, personal information for the user 20, etc.). In some implementations, the display device 150 acts as a human-machine interface (HMI) that includes a graphic user interface (GUI) configured to display the image(s) as an input interface. The display device 150 can be an LED display, an OLED display, an LCD display, or the like. The input interface can be, for example, a touchscreen or touch-sensitive substrate, a mouse, a keyboard, or any sensor system configured to sense inputs made by a human user interacting with the respiratory therapy device 110.

The humidifier 160 is coupled to or integrated in the respiratory therapy device 110 and includes a reservoir 162 for storing water that can be used to humidify the pressurized air delivered from the respiratory therapy device 110. The humidifier 160 includes a one or more heating elements 164 to heat the water in the reservoir to generate water vapor. The humidifier 160 can be fluidly coupled to a water vapor inlet of the air pathway between the blower motor 114 and the air outlet 118, or can be formed in-line with the air pathway between the blower motor 114 and the air outlet 118. For example, air flow from the air inlet 116 through the blower motor 114, and then through the humidifier 160 before exiting the respiratory therapy device 110 via the air outlet 118.

While the respiratory therapy system 100 has been described herein as including each of the respiratory therapy device 110, the user interface 120, the conduit 140, the display device 150, and the humidifier 160, more or fewer components can be included in a respiratory therapy system according to implementations of the present disclosure. For example, a first alternative respiratory therapy system includes the respiratory therapy device 110, the user interface 120, and the conduit 140. As another example, a second alternative system includes the respiratory therapy device 110, the user interface 120, and the conduit 140, and the display device 150. Thus, various respiratory therapy systems can be formed using any portion or portions of the components shown and described herein and/or in combination with one or more other components.

The control system 200 includes one or more processors 202 (hereinafter, processor 202). The control system 200 is generally used to control (e.g., actuate) the various components of the system 10 and/or analyze data obtained and/or generated by the components of the system 10. The processor 202 can be a general or special purpose processor or microprocessor. While one processor 202 is illustrated in FIG. 1, the control system 200 can include any number of processors (e.g., one processor, two processors, five processors, ten processors, etc.) that can be in a single housing, or located remotely from each other. The control system 200 (or any other control system) or a portion of the control system 200 such as the processor 202 (or any other processor(s) or portion(s) of any other control system), can be used to carry out one or more steps of any of the methods described and/or claimed herein. The control system 200 can be coupled to and/or positioned within, for example, a housing of the user device 260, a portion (e.g., the respiratory therapy device 110) of the respiratory therapy system 100, and/or within a housing of one or more of the sensors 210. The control system 200 can be centralized (within one such housing) or decentralized (within two or more of such housings, which are physically distinct). In such implementations including two or more housings containing the control system 200, the housings can be located proximately and/or remotely from each other.

The memory device 204 stores machine-readable instructions that are executable by the processor 202 of the control system 200. The memory device 204 can be any suitable computer readable storage device or media, such as, for example, a random or serial access memory device, a hard drive, a solid state drive, a flash memory device, etc. While one memory device 204 is shown in FIG. 1, the system 10 can include any suitable number of memory devices 204 (e.g., one memory device, two memory devices, five memory devices, ten memory devices, etc.). The memory device 204 can be coupled to and/or positioned within a housing of a respiratory therapy device 110 of the respiratory therapy system 100, within a housing of the user device 260, within a housing of one or more of the sensors 210, or any combination thereof. Like the control system 200, the memory device 204 can be centralized (within one such housing) or decentralized (within two or more of such housings, which are physically distinct).

In some implementations, the memory device 204 stores a user profile associated with the user. The user profile can include, for example, demographic information associated with the user, biometric information associated with the user, medical information associated with the user, self-reported user feedback, sleep parameters associated with the user (e.g., sleep-related parameters recorded from one or more earlier sleep sessions), or any combination thereof. The demographic information can include, for example, information indicative of an age of the user, a gender of the user, a race of the user, a geographic location of the user, a relationship status, a family history of insomnia or sleep apnea, an employment status of the user, an educational status of the user, a socioeconomic status of the user, or any combination thereof. The medical information can include, for example, information indicative of one or more medical conditions associated with the user, medication usage by the user, or both. The medical information data can further include a multiple sleep latency test (MSLT) result or score and/or a Pittsburgh Sleep Quality Index (PSQI) score or value. The self-reported user feedback can include information indicative of a self-reported subjective sleep score (e.g., poor, average, excellent), a self-reported subjective stress level of the user, a self-reported subjective fatigue level of the user, a self-reported subjective health status of the user, a recent life event experienced by the user, or any combination thereof.

As described herein, the processor 202 and/or memory device 204 can receive data (e.g., physiological data and/or audio data) from the one or more sensors 210 such that the data for storage in the memory device 204 and/or for analysis by the processor 202. The processor 202 and/or memory device 204 can communicate with the one or more sensors 210 using a wired connection or a wireless connection (e.g., using an RF communication protocol, a Wi-Fi communication protocol, a Bluetooth communication protocol, over a cellular network, etc.). In some implementations, the system 10 can include an antenna, a receiver (e.g., an RF receiver), a transmitter (e.g., an RF transmitter), a transceiver, or any combination thereof. Such components can be coupled to or integrated a housing of the control system 200 (e.g., in the same housing as the processor 202 and/or memory device 204), or the user device 260.

Referring to FIG. 1, the one or more sensors 210 include a pressure sensor 212, a flow rate sensor 214, temperature sensor 216, a motion sensor 218, a microphone 220, a speaker 222, a radio-frequency (RF) receiver 226, a RF transmitter 228, a camera 232, an infrared sensor 234, a photoplethysmogram (PPG) sensor 236, an electrocardiogram (ECG) sensor 238, an electroencephalography (EEG) sensor 240, a capacitive sensor 242, a force sensor 244, a strain gauge sensor 246, an electromyography (EMG) sensor 248, an oxygen sensor 250, an analyte sensor 252, a moisture sensor 254, a LiDAR sensor 256, or any combination thereof. Generally, each of the one or more sensors 210 are configured to output sensor data that is received and stored in the memory device 204 or one or more other memory devices.

While the one or more sensors 210 are shown and described as including each of the pressure sensor 212, the flow rate sensor 214, the temperature sensor 216, the motion sensor 218, the microphone 220, the speaker 222, the RF receiver 226, the RF transmitter 228, the camera 232, the infrared sensor 234, the photoplethysmogram (PPG) sensor 236, the electrocardiogram (ECG) sensor 238, the electroencephalography (EEG) sensor 240, the capacitive sensor 242, the force sensor 244, the strain gauge sensor 246, the electromyography (EMG) sensor 248, the oxygen sensor 250, the analyte sensor 252, the moisture sensor 254, and the LiDAR sensor 256, more generally, the one or more sensors 210 can include any combination and any number of each of the sensors described and/or shown herein.

As described herein, the system 10 generally can be used to generate physiological data associated with a user (e.g., a user of the respiratory therapy system 100) during a sleep session. The physiological data can be analyzed to generate one or more sleep-related parameters, which can include any parameter, measurement, etc. related to the user during the sleep session. The one or more sleep-related parameters that can be determined for the user 20 during the sleep session include, for example, an Apnea-Hypopnea Index (AHI) score, a sleep score, a flow signal, a respiration signal, a respiration rate, an inspiration amplitude, an expiration amplitude, an inspiration-expiration ratio, a number of events per hour, a pattern of events, a stage, pressure settings of the respiratory therapy device 110, a heart rate, a heart rate variability, movement of the user 20, temperature, EEG activity, EMG activity, arousal, snoring, choking, coughing, whistling, wheezing, or any combination thereof.

The one or more sensors 210 can be used to generate, for example, physiological data, audio data, or both. Physiological data generated by one or more of the sensors 210 can be used by the control system 200 to determine a sleep-wake signal associated with the user 20 (FIG. 2) during the sleep session and one or more sleep-related parameters. The sleep-wake signal can be indicative of one or more sleep states, including wakefulness, relaxed wakefulness, micro-awakenings, or distinct sleep stages such as, for example, a rapid eye movement (REM) stage, a first non-REM stage (often referred to as “N1”), a second non-REM stage (often referred to as “N2”), a third non-REM stage (often referred to as “N3”), or any combination thereof. Methods for determining sleep states and/or sleep stages from physiological data generated by one or more sensors, such as the one or more sensors 210, are described in, for example, WO 2014/047310, U.S. Patent Pub. No. 2014/0088373, WO 2017/132726, WO 2019/122413, WO 2019/122414, and U.S. Patent Pub. No. 2020/0383580 each of which is hereby incorporated by reference herein in its entirety.

In some implementations, the sleep-wake signal described herein can be timestamped to indicate a time that the user enters the bed, a time that the user exits the bed, a time that the user attempts to fall asleep, etc. The sleep-wake signal can be measured by the one or more sensors 210 during the sleep session at a predetermined sampling rate, such as, for example, one sample per second, one sample per 30 seconds, one sample per minute, etc. In some implementations, the sleep-wake signal can also be indicative of a respiration signal, a respiration rate, an inspiration amplitude, an expiration amplitude, an inspiration-expiration ratio, a number of events per hour, a pattern of events, pressure settings of the respiratory therapy device 110, or any combination thereof during the sleep session. The event(s) can include snoring, apneas, central apneas, obstructive apneas, mixed apneas, hypopneas, a mask leak (e.g., from the user interface 120), a restless leg, a sleeping disorder, choking, an increased heart rate, labored breathing, an asthma attack, an epileptic episode, a seizure, or any combination thereof. The one or more sleep-related parameters that can be determined for the user during the sleep session based on the sleep-wake signal include, for example, a total time in bed, a total sleep time, a sleep onset latency, a wake-after-sleep-onset parameter, a sleep efficiency, a fragmentation index, or any combination thereof. As described in further detail herein, the physiological data and/or the sleep-related parameters can be analyzed to determine one or more sleep-related scores.

Physiological data and/or audio data generated by the one or more sensors 210 can also be used to determine a respiration signal associated with a user during a sleep session. The respiration signal is generally indicative of respiration or breathing of the user during the sleep session. The respiration signal can be indicative of and/or analyzed to determine (e.g., using the control system 200) one or more sleep-related parameters, such as, for example, a respiration rate, a respiration rate variability, an inspiration amplitude, an expiration amplitude, an inspiration-expiration ratio, an occurrence of one or more events, a number of events per hour, a pattern of events, a sleep state, a sleet stage, an apnea-hypopnea index (AHI), pressure settings of the respiratory therapy device 110, or any combination thereof. The one or more events can include snoring, apneas, central apneas, obstructive apneas, mixed apneas, hypopneas, a mask leak (e.g., from the user interface 120), a cough, a restless leg, a sleeping disorder, choking, an increased heart rate, labored breathing, an asthma attack, an epileptic episode, a seizure, increased blood pressure, or any combination thereof. Many of the described sleep-related parameters are physiological parameters, although some of the sleep-related parameters can be considered to be non-physiological parameters. Other types of physiological and/or non-physiological parameters can also be determined, either from the data from the one or more sensors 210, or from other types of data.

The pressure sensor 212 outputs pressure data that can be stored in the memory device 204 and/or analyzed by the processor 202 of the control system 200. In some implementations, the pressure sensor 212 is an air pressure sensor (e.g., barometric pressure sensor) that generates sensor data indicative of the respiration (e.g., inhaling and/or exhaling) of the user of the respiratory therapy system 100 and/or ambient pressure. In such implementations, the pressure sensor 212 can be coupled to or integrated in the respiratory therapy device 110. The pressure sensor 212 can be, for example, a capacitive sensor, an electromagnetic sensor, a piezoelectric sensor, a strain-gauge sensor, an optical sensor, a potentiometric sensor, or any combination thereof.

The flow rate sensor 214 outputs flow rate data that can be stored in the memory device 204 and/or analyzed by the processor 202 of the control system 200. Examples of flow rate sensors (such as, for example, the flow rate sensor 214) are described in International Publication No. WO 2012/012835 and U.S. Pat. No. 10,328,219, both of which are hereby incorporated by reference herein in their entireties. In some implementations, the flow rate sensor 214 is used to determine an air flow rate from the respiratory therapy device 110, an air flow rate through the conduit 140, an air flow rate through the user interface 120, or any combination thereof. In such implementations, the flow rate sensor 214 can be coupled to or integrated in the respiratory therapy device 110, the user interface 120, or the conduit 140. The flow rate sensor 214 can be a mass flow rate sensor such as, for example, a rotary flow meter (e.g., Hall effect flow meters), a turbine flow meter, an orifice flow meter, an ultrasonic flow meter, a hot wire sensor, a vortex sensor, a membrane sensor, or any combination thereof. In some implementations, the flow rate sensor 214 is configured to measure a vent flow (e.g., intentional “leak”), an unintentional leak (e.g., mouth leak and/or mask leak), a patient flow (e.g., air into and/or out of lungs), or any combination thereof. In some implementations, the flow rate data can be analyzed to determine cardiogenic oscillations of the user. In some examples, the pressure sensor 212 can be used to determine a blood pressure of a user.

The temperature sensor 216 outputs temperature data that can be stored in the memory device 204 and/or analyzed by the processor 202 of the control system 200. In some implementations, the temperature sensor 216 generates temperatures data indicative of a core body temperature of the user 20 (FIG. 2), a skin temperature of the user 20, a temperature of the air flowing from the respiratory therapy device 110 and/or through the conduit 140, a temperature in the user interface 120, an ambient temperature, or any combination thereof. The temperature sensor 216 can be, for example, a thermocouple sensor, a thermistor sensor, a silicon band gap temperature sensor or semiconductor-based sensor, a resistance temperature detector, or any combination thereof.

The motion sensor 218 outputs motion data that can be stored in the memory device 204 and/or analyzed by the processor 202 of the control system 200. The motion sensor 218 can be used to detect movement of the user 20 during the sleep session, and/or detect movement of any of the components of the respiratory therapy system 100, such as the respiratory therapy device 110, the user interface 120, or the conduit 140. The motion sensor 218 can include one or more inertial sensors, such as accelerometers, gyroscopes, and magnetometers. In some implementations, the motion sensor 218 alternatively or additionally generates one or more signals representing bodily movement of the user, from which may be obtained a signal representing a sleep state of the user; for example, via a respiratory movement of the user. In some implementations, the motion data from the motion sensor 218 can be used in conjunction with additional data from another one of the sensors 210 to determine the sleep state of the user.

The microphone 220 outputs sound and/or audio data that can be stored in the memory device 204 and/or analyzed by the processor 202 of the control system 200. The audio data generated by the microphone 220 is reproducible as one or more sound(s) during a sleep session (e.g., sounds from the user 20). The audio data form the microphone 220 can also be used to identify (e.g., using the control system 200) an event experienced by the user during the sleep session, as described in further detail herein. The microphone 220 can be coupled to or integrated in the respiratory therapy device 110, the user interface 120, the conduit 140, or the user device 260. In some implementations, the system 10 includes a plurality of microphones (e.g., two or more microphones and/or an array of microphones with beamforming) such that sound data generated by each of the plurality of microphones can be used to discriminate the sound data generated by another of the plurality of microphones

The speaker 222 outputs sound waves that are audible to a user of the system 10 (e.g., the user 20 of FIG. 2). The speaker 222 can be used, for example, as an alarm clock or to play an alert or message to the user 20 (e.g., in response to an event). In some implementations, the speaker 222 can be used to communicate the audio data generated by the microphone 220 to the user. The speaker 222 can be coupled to or integrated in the respiratory therapy device 110, the user interface 120, the conduit 140, or the user device 260.

The microphone 220 and the speaker 222 can be used as separate devices. In some implementations, the microphone 220 and the speaker 222 can be combined into an acoustic sensor 224 (e.g., a SONAR sensor), as described in, for example, WO 2018/050913, WO 2020/104465, U.S. Pat. App. Pub. No. 2022/0007965, each of which is hereby incorporated by reference herein in its entirety. In such implementations, the speaker 222 generates or emits sound waves at a predetermined interval and the microphone 220 detects the reflections of the emitted sound waves from the speaker 222. The sound waves generated or emitted by the speaker 222 have a frequency that is not audible to the human ear (e.g., below 20 Hz or above around 18 kHz) so as not to disturb the sleep of the user 20 or the bed partner 30 (FIG. 2). Based at least in part on the data from the microphone 220 and/or the speaker 222, the control system 200 can determine a location of the user 20 (FIG. 2) and/or one or more of the sleep-related parameters described in herein such as, for example, a respiration signal, a respiration rate, an inspiration amplitude, an expiration amplitude, an inspiration-expiration ratio, a number of events per hour, a pattern of events, a sleep state, a sleep stage, pressure settings of the respiratory therapy device 110, or any combination thereof. In such a context, a sonar sensor may be understood to concern an active acoustic sensing, such as by generating and/or transmitting ultrasound and/or low frequency ultrasound sensing signals (e.g., in a frequency range of about 17-23 kHz, 18-22 kHz, or 17-18 kHz, for example), through the air.

In some implementations, the sensors 210 include (i) a first microphone that is the same as, or similar to, the microphone 220, and is integrated in the acoustic sensor 224 and (ii) a second microphone that is the same as, or similar to, the microphone 220, but is separate and distinct from the first microphone that is integrated in the acoustic sensor 224.

The RF transmitter 228 generates and/or emits radio waves having a predetermined frequency and/or a predetermined amplitude (e.g., within a high frequency band, within a low frequency band, long wave signals, short wave signals, etc.). The RF receiver 226 detects the reflections of the radio waves emitted from the RF transmitter 228, and this data can be analyzed by the control system 200 to determine a location of the user and/or one or more of the sleep-related parameters described herein. An RF receiver (either the RF receiver 226 and the RF transmitter 228 or another RF pair) can also be used for wireless communication between the control system 200, the respiratory therapy device 110, the one or more sensors 210, the user device 260, or any combination thereof. While the RF receiver 226 and RF transmitter 228 are shown as being separate and distinct elements in FIG. 1, in some implementations, the RF receiver 226 and RF transmitter 228 are combined as a part of an RF sensor 230 (e.g. a RADAR sensor). In some such implementations, the RF sensor 230 includes a control circuit. The format of the RF communication can be Wi-Fi, Bluetooth, or the like.

In some implementations, the RF sensor 230 is a part of a mesh system. One example of a mesh system is a Wi-Fi mesh system, which can include mesh nodes, mesh router(s), and mesh gateway(s), each of which can be mobile/movable or fixed. In such implementations, the Wi-Fi mesh system includes a Wi-Fi router and/or a Wi-Fi controller and one or more satellites (e.g., access points), each of which include an RF sensor that the is the same as, or similar to, the RF sensor 230. The Wi-Fi router and satellites continuously communicate with one another using Wi-Fi signals. The Wi-Fi mesh system can be used to generate motion data based on changes in the Wi-Fi signals (e.g., differences in received signal strength) between the router and the satellite(s) due to an object or person moving partially obstructing the signals. The motion data can be indicative of motion, breathing, heart rate, gait, falls, behavior, etc., or any combination thereof.

The camera 232 outputs image data reproducible as one or more images (e.g., still images, video images, thermal images, or any combination thereof) that can be stored in the memory device 204. The image data from the camera 232 can be used by the control system 200 to determine one or more of the sleep-related parameters described herein, such as, for example, one or more events (e.g., periodic limb movement or restless leg syndrome), a respiration signal, a respiration rate, an inspiration amplitude, an expiration amplitude, an inspiration-expiration ratio, a number of events per hour, a pattern of events, a sleep state, a sleep stage, or any combination thereof. Further, the image data from the camera 232 can be used to, for example, identify a location of the user, to determine chest movement of the user (FIG. 2), to determine air flow of the mouth and/or nose of the user, to determine a time when the user enters the bed (FIG. 2), and to determine a time when the user exits the bed. In some implementations, the camera 232 includes a wide angle lens or a fish eye lens.

The infrared (IR) sensor 234 outputs infrared image data reproducible as one or more infrared images (e.g., still images, video images, or both) that can be stored in the memory device 204. The infrared data from the IR sensor 234 can be used to determine one or more sleep-related parameters during a sleep session, including a temperature of the user 20 and/or movement of the user 20. The IR sensor 234 can also be used in conjunction with the camera 232 when measuring the presence, location, and/or movement of the user 20. The IR sensor 234 can detect infrared light having a wavelength between about 700 nm and about 1 mm, for example, while the camera 232 can detect visible light having a wavelength between about 380 nm and about 740 nm.

The PPG sensor 236 outputs physiological data associated with the user 20 (FIG. 2) that can be used to determine one or more sleep-related parameters, such as, for example, a heart rate, a heart rate variability, a cardiac cycle, respiration rate, an inspiration amplitude, an expiration amplitude, an inspiration-expiration ratio, estimated blood pressure parameter(s), or any combination thereof. The PPG sensor 236 can be worn by the user 20, embedded in clothing and/or fabric that is worn by the user 20, embedded in and/or coupled to the user interface 120 and/or its associated headgear (e.g., straps, etc.), etc.

The gold standard for diagnosing sleep apnea is polysomnography (PSG). PSG utilizes multiple sensing channels including electroencephalography (EEG), electrooculography (EOG), electromyography (EMG), electrocardiogramand pulse oximetry, as well as airflow and respiratory effort to assess underlying causes of sleep disturbances. However, due to the inconvenience of performing an in-lab PSG test and in part due to the global COVID pandemic, Home Sleep Apnea Testing (HSAT) based on peripheral arterial tonometry (PAT) has rapidly gained popularity and currently comprise the most widely deployed category of HSAT. Peripheral arterial tonometry-based HSATs obtain most of its sensing modalities from finger photoplethysmography (PPG, which may be the same or similar to PPG sensor 236), from which it derives the blood oxygen saturation (SpO2), pulse rate (PR), and peripheral arterial tonometry. Peripheral arterial tonometry-based HSATs allow for minimally invasive multi-night testing and are available in a fully disposable format. An example of such a system is called NightOwl™, which was described by Massie et al. (“An evaluation of the Night Owl home sleep apnea testing system,” Journal of Clinical Sleep Medicine, vol. 14, no. 10, pp. 1791-1796, October 2018, doi:10.5664/jcsm.7398). It includes a finger probe of the size of a fingertip that senses peripheral arterial tone, together with actigraphy and oximetry, and works with cloud-based analysis software. The analysis determines respiratory-related information, including occurrence of respiratory events (such as obstructive and central apnea events). The device and analyses are described in US2020/0015737A1, WO2021260190A1, and WO2021260192A1, each of which is incorporated herein in its entirety. Not only is such a device suitable for monitoring, screening and diagnosing sleep apnea, the device may be used to determine, or derive from the peripheral arterial tone signal determined from the PPG signal, for example, a respiration rate, heart rate, heart rate variability, limb and/or body motion, and from which a user's stress level may be inferred. Furthermore, the peripheral arterial tone signal rises and falls with changes in the sympathetic nervous system and thus may be used to monitor sympathetic nervous system activity as an indicator of user stress levels. Such functionality, in the form of a NightOwl™ or similar device, may be incorporated into the electronic module described herein.

The ECG sensor 238 outputs physiological data associated with electrical activity of the heart of the user 20. In some implementations, the ECG sensor 238 includes one or more electrodes that are positioned on or around a portion of the user 20 during the sleep session. The physiological data from the ECG sensor 238 can be used, for example, to determine one or more of the sleep-related parameters described herein.

The EEG sensor 240 outputs physiological data associated with electrical activity of the brain of the user 20. In some implementations, the EEG sensor 240 includes one or more electrodes that are positioned on or around the scalp of the user 20 during the sleep session. The physiological data from the EEG sensor 240 can be used, for example, to determine a sleep state and/or a sleep stage of the user 20 at any given time during the sleep session. In some implementations, the EEG sensor 240 can be integrated in the user interface 120 and/or the associated headgear (e.g., straps, etc.).

The capacitive sensor 242, the force sensor 244, and the strain gauge sensor 246 output data that can be stored in the memory device 204 and used/analyzed by the control system 200 to determine, for example, one or more of the sleep-related parameters described herein. The EMG sensor 248 outputs physiological data associated with electrical activity produced by one or more muscles. The oxygen sensor 250 outputs oxygen data indicative of an oxygen concentration of gas (e.g., in the conduit 140 or at the user interface 120). The oxygen sensor 250 can be, for example, an ultrasonic oxygen sensor, an electrical oxygen sensor, a chemical oxygen sensor, an optical oxygen sensor, a pulse oximeter (e.g., SpO2 sensor), or any combination thereof.

The analyte sensor 252 can be used to detect the presence of an analyte in the exhaled breath of the user 20. The data output by the analyte sensor 252 can be stored in the memory device 204 and used by the control system 200 to determine the identity and concentration of any analytes in the breath of the user. In some implementations, the analyte sensor 174 is positioned near a mouth of the user to detect analytes in breath exhaled from the user's mouth. For example, when the user interface 120 is a facial mask that covers the nose and mouth of the user, the analyte sensor 252 can be positioned within the facial mask to monitor the user's mouth breathing. In other implementations, such as when the user interface 120 is a nasal mask or a nasal pillow mask, the analyte sensor 252 can be positioned near the nose of the user to detect analytes in breath exhaled through the user's nose. In still other implementations, the analyte sensor 252 can be positioned near the user's mouth when the user interface 120 is a nasal mask or a nasal pillow mask. In this implementation, the analyte sensor 252 can be used to detect whether any air is inadvertently leaking from the user's mouth and/or the user interface 120. In some implementations, the analyte sensor 252 is a volatile organic compound (VOC) sensor that can be used to detect carbon-based chemicals or compounds. In some implementations, the analyte sensor 174 can also be used to detect whether the user is breathing through their nose or mouth. For example, if the data output by an analyte sensor 252 positioned near the mouth of the user or within the facial mask (e.g., in implementations where the user interface 120 is a facial mask) detects the presence of an analyte, the control system 200 can use this data as an indication that the user is breathing through their mouth.

The moisture sensor 254 outputs data that can be stored in the memory device 204 and used by the control system 200. The moisture sensor 254 can be used to detect moisture in various areas surrounding the user (e.g., inside the conduit 140 or the user interface 120, near the user's face, near the connection between the conduit 140 and the user interface 120, near the connection between the conduit 140 and the respiratory therapy device 110, etc.). Thus, in some implementations, the moisture sensor 254 can be coupled to or integrated in the user interface 120 or in the conduit 140 to monitor the humidity of the pressurized air from the respiratory therapy device 110. In other implementations, the moisture sensor 254 is placed near any area where moisture levels need to be monitored. The moisture sensor 254 can also be used to monitor the humidity of the ambient environment surrounding the user, for example, the air inside the bedroom.

The Light Detection and Ranging (LiDAR) sensor 256 can be used for depth sensing. This type of optical sensor (e.g., laser sensor) can be used to detect objects and build three dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having a LiDAR sensor 256 can measure and map an area extending 5 meters or more away from the sensor. The LiDAR data can be fused with point cloud data estimated by an electromagnetic RADAR sensor, for example. The LiDAR sensor(s) 256 can also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down, or falls down, for example. LiDAR may be used to form a 3D mesh representation of an environment. In a further use, for solid surfaces through which radio waves pass (e.g., radio-translucent materials), the LiDAR may reflect off such surfaces, thus allowing a classification of different type of obstacles.

In some implementations, the one or more sensors 210 also include a galvanic skin response (GSR) sensor, a blood flow sensor, a respiration sensor, a pulse sensor, a sphygmomanometer sensor, an oximetry sensor, a sonar sensor, a RADAR sensor, a blood glucose sensor, a color sensor, a pH sensor, an air quality sensor, a tilt sensor, a rain sensor, a soil moisture sensor, a water flow sensor, an alcohol sensor, or any combination thereof.

While shown separately in FIG. 1, any combination of the one or more sensors 210 can be integrated in and/or coupled to any one or more of the components of the system 10, including the respiratory therapy device 110, the user interface 120, the conduit 140, the humidifier 160, the control system 200, the user device 260, the activity tracker 270, the head-worn assembly 290 (e.g., an electronics module of the head-worn assembly 290), or any combination thereof. For example, the microphone 220 and the speaker 222 can be integrated in and/or coupled to the user device 260 and the pressure sensor 212 and/or flow rate sensor 132 are integrated in and/or coupled to the respiratory therapy device 110. In some implementations, at least one of the one or more sensors 210 is not coupled to the respiratory therapy device 110, the control system 200, or the user device 260, and is positioned generally adjacent to the user 20 during the sleep session (e.g., positioned on or in contact with a portion of the user 20, worn by the user 20, coupled to or positioned on the nightstand, coupled to the mattress, coupled to the ceiling, etc.).

One or more of the respiratory therapy device 110, the user interface 120, the conduit 140, the display device 150, and the humidifier 160 can contain one or more sensors (e.g., a pressure sensor, a flow rate sensor, or more generally any of the other sensors 210 described herein). These one or more sensors can be used, for example, to measure the air pressure and/or flow rate of pressurized air supplied by the respiratory therapy device 110.

The data from the one or more sensors 210 can be analyzed (e.g., by the control system 200) to determine one or more sleep-related parameters, which can include a respiration signal, a respiration rate, a respiration pattern, an inspiration amplitude, an expiration amplitude, an inspiration-expiration ratio, an occurrence of one or more events, a number of events per hour, a pattern of events, a sleep state, an apnea-hypopnea index (AHI), or any combination thereof. The one or more events can include snoring, apneas, central apneas, obstructive apneas, mixed apneas, hypopneas, a mask leak, a cough, a restless leg, a sleeping disorder, choking, an increased heart rate, labored breathing, an asthma attack, an epileptic episode, a seizure, increased blood pressure, or any combination thereof. Many of these sleep-related parameters are physiological parameters, although some of the sleep-related parameters can be considered to be non-physiological parameters. Other types of physiological and non-physiological parameters can also be determined, either from the data from the one or more sensors 210, or from other types of data.

The user device 260 (FIG. 1) includes a display device 262. The user device 260 can be, for example, a mobile device such as a smart phone, a tablet, a gaming console, a smart watch, a laptop, or the like. Alternatively, the user device 260 can be an external sensing system, a television (e.g., a smart television) or another smart home device (e.g., a smart speaker(s) such as Google Home, Amazon Echo, Alexa etc.). In some implementations, the user device is a wearable device (e.g., a smart watch). The display device 262 is generally used to display image(s) including still images, video images, or both. In some implementations, the display device 262 acts as a human-machine interface (HMI) that includes a graphic user interface (GUI) configured to display the image(s) and an input interface. The display device 262 can be an LED display, an OLED display, an LCD display, or the like. The input interface can be, for example, a touchscreen or touch-sensitive substrate, a mouse, a keyboard, or any sensor system configured to sense inputs made by a human user interacting with the user device 260. In some implementations, one or more user devices can be used by and/or included in the system 10.

In some implementations, the system 100 also includes an activity tracker 270. The activity tracker 270 is generally used to aid in generating physiological data associated with the user. The activity tracker 270 can include one or more of the sensors 210 described herein, such as, for example, the motion sensor 138 (e.g., one or more accelerometers and/or gyroscopes), the PPG sensor 154, and/or the ECG sensor 156. The physiological data from the activity tracker 270 can be used to determine, for example, a number of steps, a distance traveled, a number of steps climbed, a duration of physical activity, a type of physical activity, an intensity of physical activity, time spent standing, a respiration rate, an average respiration rate, a resting respiration rate, a maximum he respiration art rate, a respiration rate variability, a heart rate, an average heart rate, a resting heart rate, a maximum heart rate, a heart rate variability, a number of calories burned, blood oxygen saturation, electrodermal activity (also known as skin conductance or galvanic skin response), or any combination thereof. In some implementations, the activity tracker 270 is coupled (e.g., electronically or physically) to the user device 260.

In some implementations, the activity tracker 270 is a wearable device that can be worn by the user, such as a smartwatch, a wristband, a ring, or a patch. For example, referring to FIG. 2, the activity tracker 270 is worn on a wrist of the user 20. The activity tracker 270 can also be coupled to or integrated a garment or clothing that is worn by the user. Alternatively still, the activity tracker 270 can also be coupled to or integrated in (e.g., within the same housing) the user device 260. More generally, the activity tracker 270 can be communicatively coupled with, or physically integrated in (e.g., within a housing), the control system 200, the memory device 204, the respiratory therapy system 100, and/or the user device 260.

In some implementations, the system 100 also includes a blood pressure device 280. The blood pressure device 280 is generally used to aid in generating cardiovascular data for determining one or more blood pressure measurements associated with the user 20. The blood pressure device 280 can include at least one of the one or more sensors 210 to measure, for example, a systolic blood pressure component and/or a diastolic blood pressure component.

In some implementations, the blood pressure device 280 is a sphygmomanometer including an inflatable cuff that can be worn by the user 20 and a pressure sensor (e.g., the pressure sensor 212 described herein). For example, in the example of FIG. 2, the blood pressure device 280 can be worn on an upper arm of the user 20. In such implementations where the blood pressure device 280 is a sphygmomanometer, the blood pressure device 280 also includes a pump (e.g., a manually operated bulb) for inflating the cuff. In some implementations, the blood pressure device 280 is coupled to the respiratory therapy device 110 of the respiratory therapy system 100, which in turn delivers pressurized air to inflate the cuff. More generally, the blood pressure device 280 can be communicatively coupled with, and/or physically integrated in (e.g., within a housing), the control system 200, the memory device 204, the respiratory therapy system 100, the user device 260, and/or the activity tracker 270.

In other implementations, the blood pressure device 280 is an ambulatory blood pressure monitor communicatively coupled to the respiratory therapy system 100. An ambulatory blood pressure monitor includes a portable recording device attached to a belt or strap worn by the user 20 and an inflatable cuff attached to the portable recording device and worn around an arm of the user 20. The ambulatory blood pressure monitor is configured to measure blood pressure between about every fifteen minutes to about thirty minutes over a 24-hour or a 48-hour period. The ambulatory blood pressure monitor may measure heart rate of the user 20 at the same time. These multiple readings are averaged over the 24-hour period. The ambulatory blood pressure monitor determines any changes in the measured blood pressure and heart rate of the user 20, as well as any distribution and/or trending patterns of the blood pressure and heart rate data during a sleeping period and an awakened period of the user 20. The measured data and statistics may then be communicated to the respiratory therapy system 100.

The blood pressure device 280 may be positioned external to the respiratory therapy system 100, coupled directly or indirectly to the user interface 120, coupled directly or indirectly to a headgear associated with the user interface 120, or inflatably coupled to or about a portion of the user 20. The blood pressure device 280 is generally used to aid in generating physiological data for determining one or more blood pressure measurements associated with a user, for example, a systolic blood pressure component and/or a diastolic blood pressure component. In some implementations, the blood pressure device 280 is a sphygmomanometer including an inflatable cuff that can be worn by a user and a pressure sensor (e.g., the pressure sensor 212 described herein).

In some implementations, the blood pressure device 280 is an invasive device which can continuously monitor arterial blood pressure of the user 20 and take an arterial blood sample on demand for analyzing gas of the arterial blood. In some other implementations, the blood pressure device 280 is a continuous blood pressure monitor, using a radio frequency sensor and capable of measuring blood pressure of the user 20 once very few seconds (e.g., every 3 seconds, every 5 seconds, every 7 seconds, etc.) The radio frequency sensor may use continuous wave, frequency-modulated continuous wave (FMCW with ramp chirp, triangle, sinewave), other schemes such as PSK, FSK etc., pulsed continuous wave, and/or spread in ultra wideband ranges (which may include spreading, PRN codes or impulse systems).

Head-worn assembly 290 can include an electronics module, which can itself include a processor (e.g., processor 202), a memory device (e.g., memory device 204), and one or more sensors (e.g., one or more sensors 210). In some cases, the processor, memory device, and/or one or more sensors can be located in other parts of the head-worn assembly 290, such as if a band of the head-worn assembly 290 incorporates electrodes of an EEG sensor. In some cases, the head-worn assembly 290 includes an electronics module capable of processing sensor data and outputting physiological parameters and/or a diagnosis or suspected diagnosis (e.g., a likelihood the user is suffering from OSA). In some cases, the head-worn assembly 290 includes an electronics module that merely collects sensor data and relays the sensor data to another device with minimal or no processing. For example, the electronics module can include one or more sensors, a transmitter, and a battery or energy harvesting power source. The electronics module can be coupled to a band or other material to permit the head-worn assembly 290 to be worn on the head of a user. The head-worn assembly 290 can be configured operate in at least two configurations including: a first configuration in which the head-worn assembly 290 can be worn by itself, and a second configuration, in which the head-worn assembly 290 can be coupled to (e.g., mechanically, electrically, and/or communicatively) the respiratory therapy system 100 (e.g., to the user interface 120 of the respiratory therapy system 100). In some cases, at least a portion of the head-worn assembly 290 is at least mechanically coupled to the user interface 120 (e.g., to headgear of a user interface 120). At least a portion of the head-worn assembly 290 can couple to a user interface 120 at any suitable location on the user interface 120, such as coupling to headgear, a cushion, a frame, a connector 470, or the like. In some cases, at least a portion of the head-worn assembly 290 can couple to another component of the respiratory therapy system 100, such as a conduit 140. In some cases, the head-worn assembly 290 will couple to a component of the respiratory therapy system 100 in a fashion that permits the one or more sensors of the head-worn assembly 290 to still collect sensor data (e.g., motion data, audio data, and the like) associated with the user while the user engages in a sleep session.

In some cases, the electronics module can include i) an EEG sensor; ii) an electrooculography (EOG) sensor; iii) a microphone; iv) a speaker; v) a GSR sensor; vi) a PPG sensor; or vii) any combination of i-vi. Other sensors can also be used. The head-worn assembly 290 can be used to detect or determine i) head position; ii) body position; iii) mouth breathing (e.g., via a microphone sensor); iv) mask comfort (e.g., via determination of heart rate variability or respiration rate, or stress levels detected via peripheral arterial tone signal or self-reported, etc.); v) sleep quality (e.g., via determination of sleep staging, such as from EEG, microphone, and/or accelerometer data); vi) sleep apnea (e.g., presence of sleep apnea and/or type of sleep apnea); or vii) any combination of i-vi. In some cases, the head-worn assembly 290 can facilitate understanding of positional OSA (e.g., via detection of head position and/or body position and correlation with detected apnea events).

In some cases, coupling the head-worn assembly 290 to the respiratory therapy system 100 can include electrically coupling the head-worn assembly 290 to the respiratory therapy system 100 to receive power from the respiratory therapy system 100. The received power can be used to power the electronics module during use, or can be used to charge an internal power source (e.g., internal battery, capacitor, or the like), from which the electronics module can be powered during use. In an example, the head-worn assembly 290 can be powered by and charged by the respiratory therapy system 100 when used in a second configuration, but can run off of battery power when used in a first configuration. In some cases, such an electrical coupling can include passing power through a conductor in the conduit 140 and/or a portion of the user interface 120 (e.g., a conductor in the headgear 126 and/or frame 124 of the user interface 120).

In some cases, coupling the head-worn assembly 290 to the respiratory therapy system 100 can include communicatively coupling the head-worn assembly 290 to the respiratory therapy system 100 to pass data (e.g., sensor data) between the head-worn assembly 290 and the respiratory therapy system 100 and/or an associated electronic device such as user device 260. Such a communicative coupling can be wired or wireless. In an example, head-worn assembly 290 can wirelessly couple to the respiratory therapy system 100 via a Bluetooth, NFC, RFID, or similar communication standard.

As mentioned, in some cases, the head-worn assembly 290 can be communicatively coupled to other components of system 10 that can leverage the acquired sensor data, such as to a user device 260 (e.g., a user's smartphone) or a cloud-based server.

In some cases, coupling the head-worn assembly 290 to the respiratory therapy system 100 can include mechanically coupling the head-worn assembly 290 to the respiratory therapy system 100, such as to secure the head-worn assembly 290 in place on the user's head along with a component of the respiratory therapy system 100 and/or to facilitate securing a component of the respiratory therapy system 100 on the user's head. Any suitable mechanical coupling technique can be used, such as mechanically coupling via mechanical fasteners, snap fittings, hook-and-loop fasteners, magnetic couplings, and the like. In some cases, the electronics module of the head-worn assembly 290 can be coupled to a user interface 120 directly (e.g., by magnetically coupling the electronics module to a corresponding receiving area on a frame 124 of the user interface 120). In some cases, the electronics module of the head-worn assembly 290 can be coupled to the user interface 120 indirectly, such as via a band of the head-worn assembly and/or portion of the headgear 126, which can be coupled to the user interface 120. In some cases, mechanically coupling the head-worn assembly 290 to a user interface 120 can include removing a component of the user interface 120 (e.g., a headgear component, a frame component, or the like) and replacing it with one or more components of the head-worn assembly 290.

In some cases, the head-worn assembly 290 can be used in the first configuration to collect sensor data associated with a user, such as while the user is engaging in a sleep session, which can include a duration of time in which the user is trying to get to sleep, is asleep, and/or is awakening. The head-worn assembly 290 can be used in the first configuration when the user is not making use of respiratory therapy, such as i) because the user has decided to not use their respiratory therapy at all for a given sleep session; ii) because the user temporarily decided to stop using their respiratory therapy during a sleep session; or iii) because the user has not yet started using respiratory therapy at all (e.g., has not yet been diagnosed with a sleep-related disorder). In some use cases, while used in the first configuration, the head-worn assembly 290 can obtain sensor data associated with the user that can be used to generate a respiratory therapy recommendation. The respiratory therapy recommendation can be i) a recommendation to make use of respiratory therapy or that respiratory therapy may be beneficial to the user, such as a notice that the user may suffer from a sleep-related disorder that would benefit from respiratory therapy; ii) a recommendation regarding a particular respiratory therapy system 100 to use or one or more components of a respiratory therapy system 100 to use, such as a recommendation to use a particular style, type, or model of user interface; iii) a recommendation regarding one or more parameters of the respiratory therapy system 100 to use during respiratory therapy, such as a setting of the respiratory therapy device; or iv) any combination of i-iii. In an example case, if the system 10 determines, from the sensor data acquired while the head-worn assembly 290 is used in the first configuration, that the user suffers from sleep apnea and typically breaths through the mouth while sleeping, a recommendation can be made that the user consider respiratory therapy and consider using a full face mask type user interface. In some cases, the sensor data acquired while the head-worn assembly 290 is used in the first configuration can be used to determine various physiological metrics (e.g., sleep-related physiological metrics) associated with the user, such scores associated with SDB, sleep quality, general health, and the like. In some cases, as part of, instead of, or in addition to the respiratory therapy recommendation, other warning or advice can be conveyed to the user and/or a user's caregiver (e.g., bed partner, physician, head nurse, or the like). Such other warning or advice can include a recommendation to undergo a sleep study (e.g., PSG test), a HSAT, and/or to seek an appointment with a physician.

In some cases, the head-worn assembly 290 can be used in the second configuration to collect sensor data associated with the user while the user is engaging with, and optionally receiving therapy in the form of pressurized air from, the respiratory therapy system 100. When used in the second configuration to collect sensor data, the sensor data can be leveraged for various purposes, such as to monitor physiological metrics (e.g., physiological metrics that were monitored when the head-worn assembly 290 was used in the first configuration or other physiological metrics), help titration or other adjustment of the respiratory therapy system 100 during onboarding or thereafter, or for other purposes. Further, since the head-worn assembly 290 is able to acquire data that cannot be acquired by traditional components of a respiratory therapy system 100, combination of that data with sensor data from the respiratory therapy system 100 (e.g., flow rate data from the respiratory therapy device 110) can enable new types of automations, adjustments, and metrics. For example, data from the head-worn assembly 290 can be used to identify the user's lying position, which can be leveraged, along with flow rate and/or pressure data, to help identify adjustments that may improve the efficacy of the respiratory therapy based on the user's lying position. In other examples, physiological metrics can be used to determine whether the user experiences insomnia (often evidenced by a sleep onset latency, awakenings, sleep efficiency, and/or sleep fragmentation index outside of acceptable threshold levels), which information can be leveraged to treat (such as via CBTi) the condition before and/or after the user begins using the respiratory therapy system. In some cases, sensor data collected while the head-worn assembly 290 is in the second configuration can be used to determine the efficacy of the respiratory therapy and/or any recent changes in parameters of the respiratory therapy system 100. In such or other cases, the sensor data collected while the head-worn assembly 290 is in the second configuration can be used to inform an auto-adjusting feature of a respiratory therapy system, which feature adjust the flow of pressurized air to a user's airways depending on the number, type and/or severity of sleep disorder breathing events (e.g., apneas) experienced by the user while engaging in respiratory therapy.

In some cases, the head-worn assembly 290 can be used in the second configuration to facilitate fitting the user interface 120 to the face of the user, such as by acting as part of the headgear used to secure the user interface 120 to the user's head. In such cases, the head-worn assembly 290 can optionally be used with or without collecting sensor data. When sensor data is not collected, the electronics module may be simply not used or may be removed from the remainder of the head-worn assembly 290, which can then be coupled to the user interface 120.

The head-worn assembly 290 can facilitate longitudinal monitoring of a user over a relatively long period of time, which can facilitate understanding the user's needs and patterns, and can facilitate interpretation of other sensor data acquired at a later date, whether using the head-worn assembly 290 or not. Further, by permitting a user to make use of the head-worn assembly 290 in the first configuration prior to making use of it in the second configuration, the respiratory therapy on-boarding process can be improved, as the user will already have familiarity with the head-worn assembly 290 (e.g., familiarity with the component in general, sensor data generated by the head-worn assembly 290, and/or familiarity wearing the head-worn assembly 290 during sleep). In some cases, one or more components of the respiratory therapy system 100 can be coupled to the head-worn assembly 290 in stages to permit the user to become accustomed to the sensation of the different components, optionally before supplying airflow at therapy pressures.

In some cases, the head-worn assembly 290 can be associated with one or more output devices. In some cases, the one or more output devices can be integrated into the head-worn assembly 290, such as integrated into the electronics module and/or a band of the head-worn assembly 290, although that need not always be the case (e.g., other output devices that are separate from the head-worn assembly 290 may be controlled by the head-worn assembly 290 or data therefrom). The one or more output devices (e.g., lights, speakers, scent releasing modules, electrodes, etc.) can be used to generate a stimulus, such as i) a visual stimulus; ii) a tactile stimulus; iii) an aural stimulus; iv) an electrical stimulus; v) an olfactory stimulus; or vi) a chemical stimulus; or vii) any combination of i-vi. Such as stimulus can be used to improve the user's sleep, improve the user's therapy (e.g., respiratory therapy), promote hair growth, improve skin appearance, or the like.

While the control system 200 and the memory device 204 are described and shown in FIG. 1 as being a separate and distinct component of the system 100, in some implementations, the control system 200 and/or the memory device 204 are integrated in the user device 260 and/or the respiratory therapy device 110. Alternatively, in some implementations, the control system 200 or a portion thereof (e.g., the processor 202) can be located in a cloud (e.g., integrated in a server, integrated in an Internet of Things (IOT) device, connected to the cloud, be subject to edge cloud processing, etc.), located in one or more servers (e.g., remote servers, local servers, etc., or any combination thereof.

While system 10 is shown as including all of the components described above, more or fewer components can be included in a system according to implementations of the present disclosure. For example, a first alternative system includes the control system 200, the memory device 204, and at least one of the one or more sensors 210 and does not include the respiratory therapy system 100. As another example, a second alternative system includes the control system 200, the memory device 204, at least one of the one or more sensors 210, and the user device 260. As yet another example, a third alternative system includes the control system 200, the memory device 204, the respiratory therapy system 100, at least one of the one or more sensors 210, and the user device 260. Thus, various systems can be formed using any portion or portions of the components shown and described herein and/or in combination with one or more other components.

As used herein, a sleep session can be defined in multiple ways. For example, a sleep session can be defined by an initial start time and an end time. In some implementations, a sleep session is a duration where the user is asleep, that is, the sleep session has a start time and an end time, and during the sleep session, the user does not wake until the end time. That is, any period of the user being awake is not included in a sleep session. From this first definition of sleep session, if the user wakes ups and falls asleep multiple times in the same night, each of the sleep intervals separated by an awake interval is a sleep session.

Alternatively, in some implementations, a sleep session has a start time and an end time, and during the sleep session, the user can wake up, without the sleep session ending, so long as a continuous duration that the user is awake is below an awake duration threshold. The awake duration threshold can be defined as a percentage of a sleep session. The awake duration threshold can be, for example, about twenty percent of the sleep session, about fifteen percent of the sleep session duration, about ten percent of the sleep session duration, about five percent of the sleep session duration, about two percent of the sleep session duration, etc., or any other threshold percentage. In some implementations, the awake duration threshold is defined as a fixed amount of time, such as, for example, about one hour, about thirty minutes, about fifteen minutes, about ten minutes, about five minutes, about two minutes, etc., or any other amount of time.

In some implementations, a sleep session is defined as the entire time between the time in the evening at which the user first entered the bed, and the time the next morning when user last left the bed. Put another way, a sleep session can be defined as a period of time that begins on a first date (e.g., Monday, Jan. 6, 2020) at a first time (e.g., 10:00 PM), that can be referred to as the current evening, when the user first enters a bed with the intention of going to sleep (e.g., not if the user intends to first watch television or play with a smart phone before going to sleep, etc.), and ends on a second date (e.g., Tuesday, Jan. 7, 2020) at a second time (e.g., 7:00 AM), that can be referred to as the next morning, when the user first exits the bed with the intention of not going back to sleep that next morning.

In some implementations, the user can manually define the beginning of a sleep session and/or manually terminate a sleep session. For example, the user can select (e.g., by clicking or tapping) one or more user-selectable element that is displayed on the display device 262 of the user device 260 (FIG. 1) to manually initiate or terminate the sleep session.

Generally, the sleep session includes any point in time after the user 20 has laid or sat down in the bed 40 (or another area or object on which they intend to sleep), and has turned on the respiratory therapy device 110 and donned the user interface 120. The sleep session can thus include time periods (i) when the user 20 is using the respiratory therapy system 100, but before the user 20 attempts to fall asleep (for example when the user 20 lays in the bed 40 reading a book); (ii) when the user 20 begins trying to fall asleep but is still awake; (iii) when the user 20 is in a light sleep (also referred to as stage 1 and stage 2 of non-rapid eye movement (NREM) sleep); (iv) when the user 20 is in a deep sleep (also referred to as slow-wave sleep, SWS, or stage 3 of NREM sleep); (v) when the user 20 is in rapid eye movement (REM) sleep; (vi) when the user 20 is periodically awake between light sleep, deep sleep, or REM sleep; or (vii) when the user 20 wakes up and does not fall back asleep.

The sleep session is generally defined as ending once the user 20 removes the user interface 120, turns off the respiratory therapy device 110, and gets out of bed 40. In some implementations, the sleep session can include additional periods of time, or can be limited to only some of the above-disclosed time periods. For example, the sleep session can be defined to encompass a period of time beginning when the respiratory therapy device 110 begins supplying the pressurized air to the airway or the user 20, ending when the respiratory therapy device 110 stops supplying the pressurized air to the airway of the user 20, and including some or all of the time points in between, when the user 20 is asleep or awake.

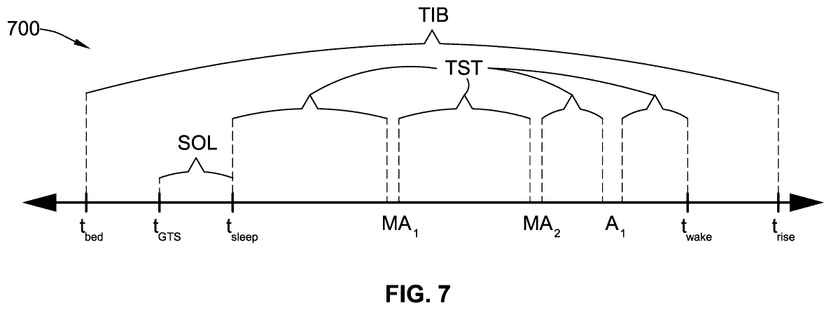

Referring to the timeline 700 in FIG. 7 the enter bed time t_{bed} is associated with the time that the user initially enters the bed (e.g., bed 40 in FIG. 2) prior to falling asleep (e.g., when the user lies down or sits in the bed). The enter bed time t_{bed} can be identified based on a bed threshold duration to distinguish between times when the user enters the bed for sleep and when the user enters the bed for other reasons (e.g., to watch TV). For example, the bed threshold duration can be at least about 10 minutes, at least about 20 minutes, at least about 30 minutes, at least about 45 minutes, at least about 1 hour, at least about 2 hours, etc. While the enter bed time t_{bed} is described herein in reference to a bed, more generally, the enter time t_{bed} can refer to the time the user initially enters any location for sleeping (e.g., a couch, a chair, a sleeping bag, etc.).

The go-to-sleep time (GTS) is associated with the time that the user initially attempts to fall asleep after entering the bed (t_{bed}). For example, after entering the bed, the user may engage in one or more activities to wind down prior to trying to sleep (e.g., reading, watching TV, listening to music, using the user device 260, etc.). The initial sleep time (tₛₗₑₑₚ) is the time that the user initially falls asleep. For example, the initial sleep time (tₛₗₑₑₚ) can be the time that the user initially enters the first non-REM sleep stage.

The wake-up time t_{wake} is the time associated with the time when the user wakes up without going back to sleep (e.g., as opposed to the user waking up in the middle of the night and going back to sleep). The user may experience one of more unconscious microawakenings (e.g., microawakenings MA1 and MA2) having a short duration (e.g., 5 seconds, 10 seconds, 30 seconds, 1 minute, etc.) after initially falling asleep. In contrast to the wake-up time t_{wake}, the user goes back to sleep after each of the microawakenings MA₁ and MA₂. Similarly, the user may have one or more conscious awakenings (e.g., awakening A) after initially falling asleep (e.g., getting up to go to the bathroom, attending to children or pets, sleep walking, etc.). However, the user goes back to sleep after the awakening A. Thus, the wake-up time t_{wake} can be defined, for example, based on a wake threshold duration (e.g., the user is awake for at least 15 minutes, at least 20 minutes, at least 30 minutes, at least 1 hour, etc.).

Similarly, the rising time tᵣᵢₛₑ is associated with the time when the user exits the bed and stays out of the bed with the intent to end the sleep session (e.g., as opposed to the user getting up during the night to go to the bathroom, to attend to children or pets, sleep walking, etc.). In other words, the rising time tᵣᵢₛₑ is the time when the user last leaves the bed without returning to the bed until a next sleep session (e.g., the following evening). Thus, the rising time tᵣᵢₛₑ can be defined, for example, based on a rise threshold duration (e.g., the user has left the bed for at least 15 minutes, at least 20 minutes, at least 30 minutes, at least 1 hour, etc.). The enter bed time t_{bed} time for a second, subsequent sleep session can also be defined based on a rise threshold duration (e.g., the user has left the bed for at least 4 hours, at least 6 hours, at least 8 hours, at least 12 hours, etc.).

As described above, the user may wake up and get out of bed one more times during the night between the initial t_{bed} and the final tᵣᵢₛₑ. In some implementations, the final wake-up time t_{wake} and/or the final rising time tᵣᵢₛₑ that are identified or determined based on a predetermined threshold duration of time subsequent to an event (e.g., falling asleep or leaving the bed). Such a threshold duration can be customized for the user. For a standard user which goes to bed in the evening, then wakes up and goes out of bed in the morning any period (between the user waking up (t_{wake}) or raising up (tᵣᵢₛₑ), and the user either going to bed (t_{bed}), going to sleep (t_{GTS}) or falling asleep (tₛₗₑₑₚ) of between about 12 and about 18 hours can be used. For users that spend longer periods of time in bed, shorter threshold periods may be used (e.g., between about 8 hours and about 14 hours). The threshold period may be initially selected and/or later adjusted based on the system monitoring the user's sleep behavior.

The total time in bed (TIB) is the duration of time between the time enter bed time t_{bed} and the rising time tᵣᵢₛₑ. The total sleep time (TST) is associated with the duration between the initial sleep time and the wake-up time, excluding any conscious or unconscious awakenings and/or micro-awakenings therebetween. Generally, the total sleep time (TST) will be shorter than the total time in bed (TIB) (e.g., one minute short, ten minutes shorter, one hour shorter, etc.). For example, referring to the timeline 700 of FIG. 7, the total sleep time (TST) spans between the initial sleep time tₛₗₑₑₚ and the wake-up time t_{wake}, but excludes the duration of the first micro-awakening MA₁, the second micro-awakening MA₂, and the awakening A. As shown, in this example, the total sleep time (TST) is shorter than the total time in bed (TIB).

In some implementations, the total sleep time (TST) can be defined as a persistent total sleep time (PTST). In such implementations, the persistent total sleep time excludes a predetermined initial portion or period of the first non-REM stage (e.g., light sleep stage). For example, the predetermined initial portion can be between about 30 seconds and about 20 minutes, between about 1 minute and about 10 minutes, between about 3 minutes and about 5 minutes, etc. The persistent total sleep time is a measure of sustained sleep, and smooths the sleep-wake hypnogram. For example, when the user is initially falling asleep, the user may be in the first non-REM stage for a very short time (e.g., about 30 seconds), then back into the wakefulness stage for a short period (e.g., one minute), and then goes back to the first non-REM stage. In this example, the persistent total sleep time excludes the first instance (e.g., about 30 seconds) of the first non-REM stage.

In some implementations, the sleep session is defined as starting at the enter bed time (t_{bed}) and ending at the rising time (tᵣᵢₛₑ), i.e., the sleep session is defined as the total time in bed (TIB). In some implementations, a sleep session is defined as starting at the initial sleep time (tₛₗₑₑₚ) and ending at the wake-up time (t_{wake}). In some implementations, the sleep session is defined as the total sleep time (TST). In some implementations, a sleep session is defined as starting at the go-to-sleep time (t_{GTS}) and ending at the wake-up time (t_{wake}). In some implementations, a sleep session is defined as starting at the go-to-sleep time (t_{GTS}) and ending at the rising time (tᵣᵢₛₑ). In some implementations, a sleep session is defined as starting at the enter bed time (t_{bed}) and ending at the wake-up time (t_{wake}). In some implementations, a sleep session is defined as starting at the initial sleep time (tₛₗₑₑₚ) and ending at the rising time (tᵣᵢₛₑ).

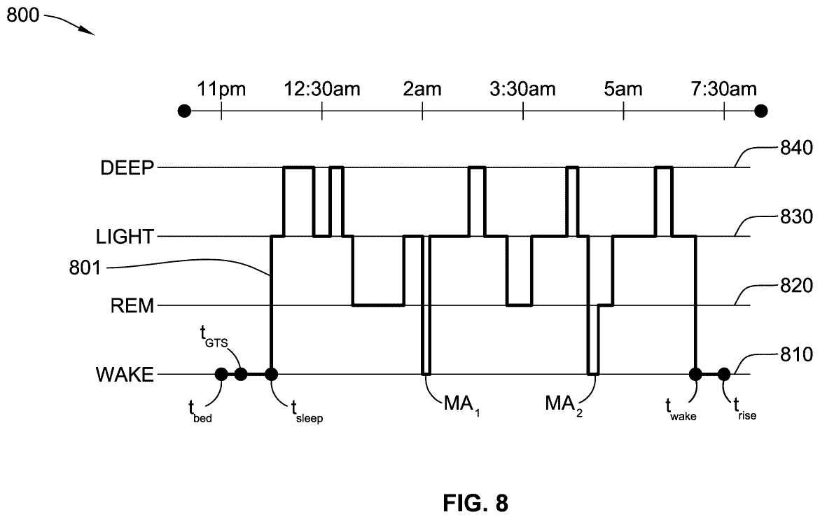

Referring to FIG. 8, an exemplary hypnogram 800 corresponding to the timeline 700 (FIG. 7), according to some implementations, is illustrated. As shown, the hypnogram 800 includes a sleep-wake signal 801, a wakefulness stage axis 810, a REM stage axis 820, a light sleep stage axis 830, and a deep sleep stage axis 840. The intersection between the sleep-wake signal 801 and one of the axes 810-840 is indicative of the sleep stage at any given time during the sleep session.

The sleep-wake signal 801 can be generated based on physiological data associated with the user (e.g., generated by one or more of the sensors 210 described herein). The sleep-wake signal can be indicative of one or more sleep states, including wakefulness, relaxed wakefulness, microawakenings, a REM stage, a first non-REM stage, a second non-REM stage, a third non-REM stage, or any combination thereof. In some implementations, one or more of the first non-REM stage, the second non-REM stage, and the third non-REM stage can be grouped together and categorized as a light sleep stage or a deep sleep stage. For example, the light sleep stage can include the first non-REM stage and the deep sleep stage can include the second non-REM stage and the third non-REM stage. While the hypnogram 800 is shown in FIG. 8 as including the light sleep stage axis 830 and the deep sleep stage axis 840, in some implementations, the hypnogram 800 can include an axis for each of the first non-REM stage, the second non-REM stage, and the third non-REM stage. In other implementations, the sleep-wake signal can also be indicative of a respiration signal, a respiration rate, an inspiration amplitude, an expiration amplitude, an inspiration-expiration ratio, a number of events per hour, a pattern of events, or any combination thereof. Information describing the sleep-wake signal can be stored in the memory device 204.

The hypnogram 800 can be used to determine one or more sleep-related parameters, such as, for example, a sleep onset latency (SOL), wake-after-sleep onset (WASO), a sleep efficiency (SE), a sleep fragmentation index, sleep blocks, or any combination thereof.

The sleep onset latency (SOL) is defined as the time between the go-to-sleep time (t_{GTS}) and the initial sleep time (tₛₗₑₑₚ). In other words, the sleep onset latency is indicative of the time that it took the user to actually fall asleep after initially attempting to fall asleep. In some implementations, the sleep onset latency is defined as a persistent sleep onset latency (PSOL). The persistent sleep onset latency differs from the sleep onset latency in that the persistent sleep onset latency is defined as the duration time between the go-to-sleep time and a predetermined amount of sustained sleep. In some implementations, the predetermined amount of sustained sleep can include, for example, at least 10 minutes of sleep within the second non-REM stage, the third non-REM stage, and/or the REM stage with no more than 2 minutes of wakefulness, the first non-REM stage, and/or movement therebetween. In other words, the persistent sleep onset latency requires up to, for example, 8 minutes of sustained sleep within the second non-REM stage, the third non-REM stage, and/or the REM stage. In other implementations, the predetermined amount of sustained sleep can include at least 10 minutes of sleep within the first non-REM stage, the second non-REM stage, the third non-REM stage, and/or the REM stage subsequent to the initial sleep time. In such implementations, the predetermined amount of sustained sleep can exclude any micro-awakenings (e.g., a ten second micro-awakening does not restart the 10-minute period).

The wake-after-sleep onset (WASO) is associated with the total duration of time that the user is awake between the initial sleep time and the wake-up time. Thus, the wake-after-sleep onset includes short and micro-awakenings during the sleep session (e.g., the micro-awakenings MA₁ and MA₂ shown in FIG. 7), whether conscious or unconscious. In some implementations, the wake-after-sleep onset (WASO) is defined as a persistent wake-after-sleep onset (PWASO) that only includes the total durations of awakenings having a predetermined length (e.g., greater than 10 seconds, greater than 30 seconds, greater than 60 seconds, greater than about 5 minutes, greater than about 10 minutes, etc.) The sleep efficiency (SE) is determined as a ratio of the total time in bed (TIB) and the total sleep time (TST). For example, if the total time in bed is 8 hours and the total sleep time is 7.5 hours, the sleep efficiency for that sleep session is 93.75%. The sleep efficiency is indicative of the sleep hygiene of the user. For example, if the user enters the bed and spends time engaged in other activities (e.g., watching TV) before sleep, the sleep efficiency will be reduced (e.g., the user is penalized). In some implementations, the sleep efficiency (SE) can be calculated based on the total time in bed (TIB) and the total time that the user is attempting to sleep. In such implementations, the total time that the user is attempting to sleep is defined as the duration between the go-to-sleep (GTS) time and the rising time described herein. For example, if the total sleep time is 8 hours (e.g., between 11 PM and 7 AM), the go-to-sleep time is 10:45 PM, and the rising time is 7:15 AM, in such implementations, the sleep efficiency parameter is calculated as about 94%.

The fragmentation index is determined based at least in part on the number of awakenings during the sleep session. For example, if the user had two micro-awakenings (e.g., micro-awakening MA₁ and micro-awakening MA₂ shown in FIG. 7), the fragmentation index can be expressed as 2. In some implementations, the fragmentation index is scaled between a predetermined range of integers (e.g., between 0 and 10).

The sleep blocks are associated with a transition between any stage of sleep (e.g., the first non-REM stage, the second non-REM stage, the third non-REM stage, and/or the REM) and the wakefulness stage. The sleep blocks can be calculated at a resolution of, for example, 30 seconds.

In some implementations, the systems and methods described herein can include generating or analyzing a hypnogram including a sleep-wake signal to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof based at least in part on the sleep-wake signal of a hypnogram.

In other implementations, one or more of the sensors 210 can be used to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof, which in turn define the sleep session. For example, the enter bed time t_{bed} can be determined based on, for example, data generated by the motion sensor 218, the microphone 220, the camera 232, or any combination thereof. The go-to-sleep time can be determined based on, for example, data from the motion sensor 218 (e.g., data indicative of no movement by the user), data from the camera 232 (e.g., data indicative of no movement by the user and/or that the user has turned off the lights) data from the microphone 220 (e.g., data indicative of the using turning off a TV), data from the user device 260 (e.g., data indicative of the user no longer using the user device 260), data from the pressure sensor 212 and/or the flow rate sensor 214 (e.g., data indicative of the user turning on the respiratory therapy device 110, data indicative of the user donning the user interface 120, etc.), or any combination thereof.

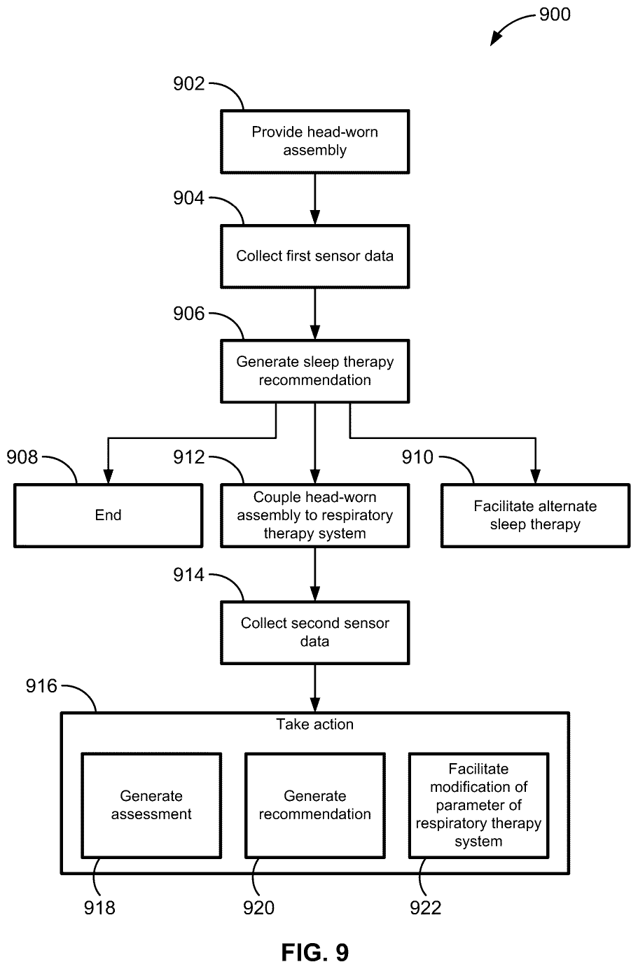

FIG. 9 is a flowchart depicting a process 900 for using a head-worn assembly according to certain aspects of the present disclosure.

At block 902, the head-worn assembly can be provided. Any suitable head-worn assembly can be used, such as head-worn assembly 290 of FIG. 1. The head-worn assembly can be provided in or can be adjusted to a first configuration, in which the head-worn assembly is wearable on the head of the user by itself. Various forms of head-worn assemblies can be used, such as headbands, eye patches, eyemasks, head coverings, or the like. Providing the head-worn assembly can include placing the head-worn assembly on the head of the user and starting data collection.

At block 904, first sensor data can be collected. The first sensor data can be sensor data acquired from one or more sensors of the head-worn assembly. In some cases, the first sensor data can additionally include additional sensor data from one or more additional sensors associated with another device, such as sensor(s) of a user device (e.g., smartphone). For example, the head-worn assembly may acquire movement data and the user's smartphone may acquire microphone data. In some cases, however, the first sensor data includes only sensor data from the sensor(s) of the head-worn assembly. The collected sensor data can be transferred from the head-worn assembly to another device for further processing, such as being transferred to a smartphone or cloud computer, although that need not always be the case. In some cases, the additional processing can occur on the head-worn assembly itself.

At block 906, a sleep therapy recommendation can be generated. Generating the sleep therapy recommendation can include analyzing the first sensor data. In some cases, analyzing the first sensor data can include determining physiological data associated with the user as disclosed in further detail herein. For example, the first sensor data can be used to generate an AHI score, a sleep score, and an indication of mouth breathing (e.g., a mouth breathing signal) associated with a sleep session of the user. In some cases, analyzing sensor data (e.g., the first sensor data) can include determining other types of physiological data, such as i) identifying one or more sleep-disorder breathing events; ii) determining a sleep quality score; iii) determining a general health score; iv) detecting head position information; v) detecting body position information; vi) identifying mouth breathing; vii) determining stress level information; viii) determining heart rate information; ix) determining respiration rate information; x) determining sympathetic nervous system activation; xi) determining apnea hypopnea index information; xii) determining sleep state information; xiii) determining sleep stage information; xiv) determining arousals; xv) detecting insomnia; xvi) determining hyperarousal; xvii) determining hypoxic burden; xviii) differentiating a central sleep apnea event from an obstructive sleep apnea event; xix) detecting restless leg syndrome; xx) detecting periodic limb movement disorder; xxi) detecting rapid-eye-movement-predominant obstructive sleep apnea; xxii) detecting positional obstructive sleep apnea; xxiii) detecting arrhythmia; xxiv) determining blood pressure; xxv) determining blood glucose level; xxvi) determining a core body temperature; or xxvii) any combination of i-xxvi.

Depending on the physiological data or other information from the first sensor data, a sleep therapy recommendation can be generated. The sleep therapy recommendation can be any recommendation associated with a possible sleep therapy of the user. In some cases, a sleep therapy recommendation can include a recommendation to i) not partake in any sleep therapy; ii) partake in sleep therapy generally; iii) partake in respiratory therapy; or iv) partake in an alternate sleep therapy other than respiratory therapy. In some cases, the sleep therapy recommendation can include a recommendation of equipment to use in respiratory therapy or parameters of a respiratory therapy system to use in respiratory therapy. In some cases, the sleep therapy recommendation can include a recommendation about when to commence sleep therapy, what actions to take prior to engaging in sleep therapy (e.g., a recommendation for good sleep hygiene, such as not using devices such as TVs or smartphones immediately prior to starting a sleep session), what actions to take after a sleep therapy session, or the like. In some cases, the sleep therapy recommendation can include a recommendation to continue using the head-worn assembly in the first configuration for additional iterations of blocks 904, 906.

After a sleep therapy recommendation is generated, it can be presented to the user or another (e.g., a caregiver associated with the user) in any suitable fashion, such as by being displayed on a display device. In some cases, the sleep therapy recommendation can include a parameter or setting that can be transmitted to the necessary recipient for automatic enactment, such as a setting on a respiratory therapy device being automatically transferred to the respiratory therapy device for implementation.

In some cases, in addition to or instead of generating the sleep therapy recommendation at block 906, one or more physiological metrics (e.g., a sleep score) can be generated and presented to the user. In some cases, a sleep therapy recommendation can be direct (e.g., a notice instructing the user to start respiratory therapy or to seek a sleep-related disorder diagnosis) or indirect (e.g., a notice displaying a sleep score that is lower than a threshold value, which may indicate to the user that sleep some therapy would be beneficial).

In some cases, such as if the sleep therapy recommendation generated at block 906 is a recommendation that no sleep therapy is needed, process 900 can end at block 908. The user can continue wearing the head-worn assembly for future sleep sessions (e.g., to continue tracking sleep-related data, such as sleep scores) and/or can otherwise dispose of the head-worn assembly.

In some cases, such as if the sleep therapy recommendation generated at block 906 is a recommendation to engage in an alternate sleep therapy other than respiratory therapy, process 900 can continue by facilitating the alternate sleep therapy at block 910. Facilitating the alternate sleep therapy can include presenting information to the user about the alternate sleep therapy, providing information to the user or a caregiver to facilitate diagnosing a sleep-related condition or developing the alternate sleep therapy, or taking any other suitable action to help facilitate the user's engagement in the alternate sleep therapy.

In some cases, however, the sleep therapy recommendation generated at block 906 can be indicative of or can otherwise include a recommendation that the user engage in respiratory therapy. The user can then procure, if one had not been procured already, a respiratory therapy system. In some cases, the sleep therapy recommendation can include information usable to help diagnose the user's sleep-related condition and/or to help determine which respiratory therapy system or components thereof should be used for the user's respiratory therapy.

At block 912, the head-worn assembly can be coupled to the respiratory therapy system. In some cases, coupling the head-worn assembly to the respiratory therapy system can include coupling the entire head-worn assembly, although that need not always be the case. In some cases, coupling the head-worn assembly to the respiratory therapy system can include first removing one or more components. In a first example, a head-worn assembly that is a headband can be coupled, in its entirety, to a user interface. In a second example, the electronics module of a head-worn assembly can be separated from a remainder of the head-worn assembly, then the electronics module can be coupled to a user interface. In a third example, the electronics module of a head-worn assembly can be separated from a band of the head-worn assembly, then the band can be coupled to a user interface. In some cases, coupling the head-worn assembly to the respiratory therapy system can include coupling the head-worn assembly to a user interface of the respiratory therapy system, although that need not always be the case.

Coupling the head-worn assembly to the respiratory therapy system can result in the head-worn assembly being used in the second configuration.

At block 914, second sensor data can be collected. Collecting second sensor data can be similar to collecting first sensor data at block 904, but while the head-worn assembly is in the second configuration. In some cases, collecting second sensor data occurs while the user is engaging in respiratory therapy (e.g., receiving pressurized air from the respiratory therapy device), although that need not always be the case. In some cases, the second sensor data can be collected while the user is wearing a user interface but not yet receiving respiratory therapy. In such cases, the user may be becoming familiar with, and used to wearing during a sleep session, the respiratory therapy system or components thereof in advance of beginning therapy.

In some cases, second sensor data can additionally include additional sensor data from one or more additional sensors associated with another device, such as sensor(s) of a user device (e.g., smartphone) and/or sensor(s) of the respiratory therapy system. In an example, sensor(s) of the respiratory therapy system may provide flow rate and/or pressure data associated with pressurized air supplied to the user engaging in a sleep session, while the head-worn assembly supplies movement data indicative of the user's lying position. Together, the flow rate and/or pressure data and movement data may be used to automatically adjust operation of the respiratory therapy device. For example, if it is determined that the user suffers from positional sleep apnea, flow rate and/or pressure settings may be adjusted, such as increased, when the user is in a lying position associated with a greater likelihood of apneas events occurring. Similarly, flow rate and/or pressure data and EEG data (or other sleep staging data) may be used to automatically adjust operation of the respiratory therapy device. For example, if it is determined suffers from REM-dominant sleep apnea, flow rate and/or pressure settings may be adjusted, such as increased, when the user is in or about to enter an REM sleep stage.

In some cases, second sensor data can be initially collected by the head-worn assembly and transmitted to the user interface and/or to a respiratory therapy device via wired or wireless techniques. In some cases, an antenna in the user interface or the respiratory therapy device can be used to wirelessly receive the second sensor data from the electronics module when the head-worn assembly is worn in the second configuration. In some cases, an electrical connector on the user interface or the respiratory therapy device can be used to receive the second sensor data by wire (e.g., via electrical connection between the electronics module and the electrical connector).

At block 916, action can be taken based on the second sensor data, and optionally further based on the first sensor data (e.g., a comparison between the first sensor data and the second sensor data). Taking the action at block 916 can include analyzing the second sensor data, such as at determine physiological data associated with the user engaging in a sleep session while wearing the user interface and/or while receiving respiratory therapy.

In some cases, taking action at block 916 can include generating an assessment at block 918. Generating the assessment can include determining a score (e.g., a sleep score and/or a therapy score (such as a my Air™ score) or other metric usable to determine whether or not the user is responding positively to the respiratory therapy. In some cases, a modification of a parameter of the respiratory therapy system may be associated with the assessment, such that the assessment is indicative of whether or not the modification of the parameter was beneficial and/or should be maintained or reversed.

In some cases, taking action at block 916 can include generating a recommendation at block 920. Generating a recommendation at block 920 can be similar to generating a sleep therapy recommendation at block 906, although other recommendations can be generated. The recommendation generated at block 920 can include a recommendation to continue use of the respiratory therapy system, a recommendation to take certain action prior to the next sleep session (e.g., to make a change to the respiratory therapy system, to make a change to the user's sleeping environment, to do or avoid doing certain actions immediately prior to sleep, etc.), or the like.

In some cases, taking action at block 916 can include facilitating modification of a parameter of the respiratory therapy system at block 922. A parameter of the respiratory therapy system can include any modifiable aspect of the respiratory therapy system, such as the style of user interface used, the type of conduit used, one or more settings of the respiratory therapy device, or the like. Facilitating modification of the parameter can include automatically modifying the parameter (e.g., automatically adjusting a setting on the respiratory therapy device during the sleep session or prior to the next sleep session), suggesting the modification (e.g., generating a notice recommending that the user switch to a different user interface), or otherwise helping the modification take place (e.g., having a different user interface automatically shipped to the user for them to try at their discretion).

In some cases, taking action at block 916 based on the second sensor data can include taking other actions. Taking action at block 916 can occur dynamically (e.g., during the sleep session in response to realtime sensor data collected during the sleep session), or delayed (e.g., taking action after a sleep session has concluded based on sensor data from the entire sleep session).

While process 900 is described with certain blocks in a certain order, in some cases process 900 can occur with fewer blocks, with more blocks, with one or more blocks replacing one or more existing blocks, with blocks being performed in different orders, or the like. For example, in some cases, instead of generating a sleep therapy recommendation at block 906, only a sleep score can be generated.

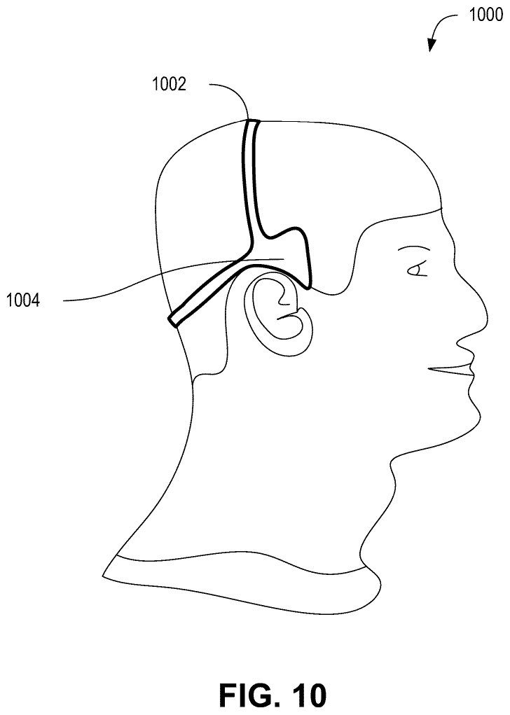

FIG. 10 is a side view of a head-worn assembly base 1002, according to certain aspects of the present disclosure. The head-worn assembly base 1002 is shown worn by a user 1000. The head-worn assembly base 1002 includes a mounting point 1004 on either side of the user's head. Here, the mounting point 1004 is located just above the user's ear, although in some cases the mounting point 1004 may be otherwise located.

The mounting point 1004 can include a mounting mechanism designed to interact with, or otherwise removably couple to, a sensor headset and/or a user interface. As depicted in FIG. 10, the mounting point 1004 includes a hook-and-loop surface designed to removably couple to a corresponding hook-and-loop surface on a sensor headset and/or a user interface. However, as described herein, any suitable coupling mechanisms can be used, such as mechanical coupling via mechanical fasteners, snap fittings, hook-and-loop fasteners, magnetic couplings, and the like.

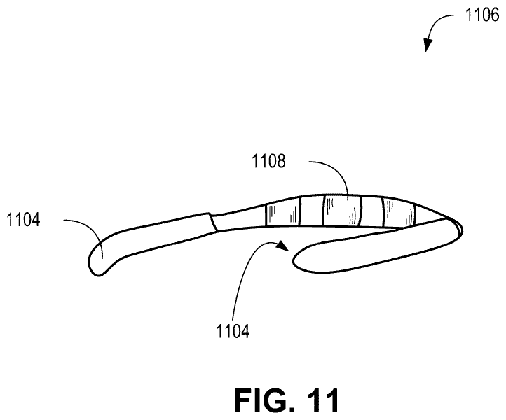

FIG. 11 is an axonometric view of a sensor headset 1106, according to certain aspects of the present disclosure. The sensor headset 1106 can include a body having one or more sensors 1108 (e.g., EEG and PPG sensors). The body of the sensor headset 1106 can include multiple attachment points 1104. The attachment points 1104 of the sensor headset 1106 can be designed to removably couple to mounting points of a head-worn assembly base (e.g., mounting point 1004 of FIG. 10). For example, the attachment points 1104 can include a hook-and-loop surface designed to couple to a corresponding hook-and-loop surface of a mounting point of a head-worn assembly base.

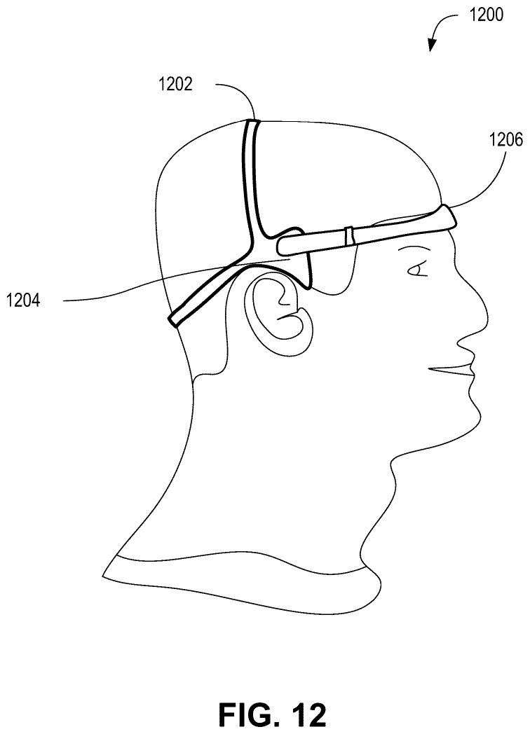

FIG. 12 is a side view of a sensor headset 1206 coupled to a head-worn assembly base 1202, according to certain aspects of the present disclosure. The head-worn assembly base 1202 can be the head-worn assembly base 1002 of FIG. 10.

The sensor headset 1206 coupled to the head-worn assembly base 1202 can effectively create a version of the head-worn assembly described in further detail herein, such as head-worn assembly 390 of FIG. 3. As depicted in FIG. 12, the sensor headset 1206 is removably coupled to the head-worn assembly base 1202 at a mounting point 1204. When worn in this fashion by the user 1200, the sensor headset 1206 can be positioned to take suitable measurements of the user 1200 to generate sensor data associated with the user 1200.

As depicted in FIG. 12, the user 1200 may be making use of the sensor headset 1206 to collect sensor data without necessarily making use of respiratory therapy, such as described herein (e.g., with reference to head-worn assembly 290 of FIG. 2).

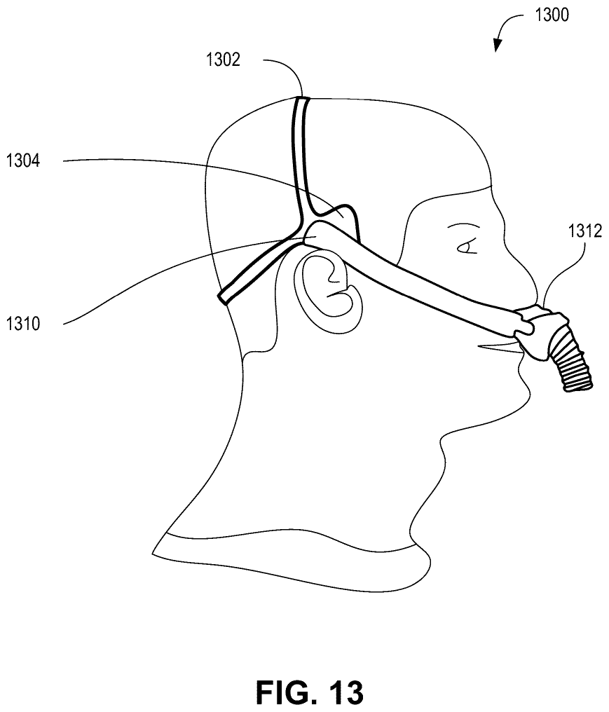

FIG. 13 is a side view of a user interface 1312 coupled to a head-worn assembly base 1302, according to certain aspects of the present disclosure. The head-worn assembly base 1302 can be the head-worn assembly base 1002 of FIG. 10.

The user interface 1312 can be coupled to the head-worn assembly base 1302 via one or more straps 1310 that removably couple to corresponding mounting points 1304 of the head-worn assembly base 1302. For example, a strap 1310 can include a hook-and-loop surface that removably couples to a corresponding hook-and-loop surface of a corresponding mounting point 1304.

As depicted in FIG. 13, the user 1300 is engaging in (or prepared to engage in) respiratory therapy, although not necessarily while using a sensor headset.

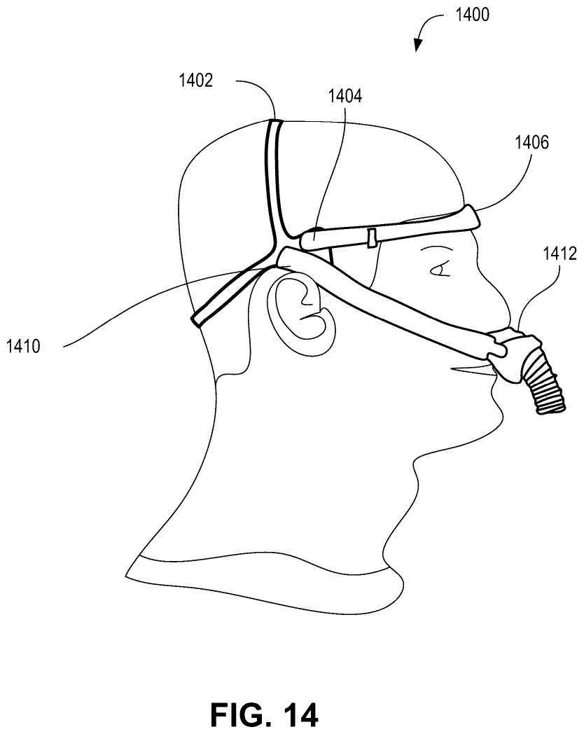

FIG. 14 is a side view of a sensor headset 1406 and a user interface 1412 both coupled to a head-worn assembly base 1402, according to certain aspects of the present disclosure. The head-worn assembly base 1402 can be the head-worn assembly base 1002 of FIG. 10. The sensor headset 1406 can be sensor headset 1106 of FIG. 11.

The sensor headset 1406 can be coupled to the head-worn assembly base 1402 in a fashion similar to that described with reference to FIGS. 11-12. The user interface 1412 can be coupled to the heard-worn assembly base 1402 in a fashion similar to that described with reference to FIG. 13. In some cases, however, one of the sensor headset 1406 and the user interface 1412 can be coupled to the head-worn assembly base 1402 via the other of the sensor headset 1406 and the user interface 1412. For example, attachment points of the sensor headset 1406 may removably couple to corresponding mounting points of the strap 1410 of the user interface 1412, and the strap 1410 of the user interface 1412 can itself removably couple to the mounting points of the head-worn assembly base 1402.

As depicted in FIG. 14, the user 1400 may be engaging in (or be prepared to engage in) respiratory therapy while also making use of the sensor headset to collect sensor data, such as described herein (e.g., with reference to head-worn assembly 291 of FIG. 2).

One or more elements or aspects or steps, or any portion(s) thereof, from one or more of any of the claims below can be combined with one or more elements or aspects or steps, or any portion(s) thereof, from one or more of any of the other claims below, or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

1. A method, comprising:
providing a head-worn assembly, the head-worn assembly having an electronics module, the electronics module including one or more sensors, the head-worn assembly being couplable to a user interface and wearable on a head of a user, wherein the head-worn assembly is wearable on the head and not coupled to the user interface when in a first configuration, and wherein the head-worn assembly is wearable on the head and coupled to the user interface when in a second configuration;
collecting first sensor data from the one or more sensors while the user wears the head-worn assembly in the first configuration during a first sleep session; and
collecting second sensor data from the one or more sensors while the user wears the head-worn assembly in the second configuration coupled to the user interface during a second sleep session.

2. The method of claim 1, further comprising using the user interface with a respiratory therapy device to provide a respiratory therapy determined based at least in part on the first sensor data.

3. (canceled)

4. The method of claim 2, further comprising selecting the user interface from a plurality of potential user interfaces based at least in part on the first sensor data.

5. The method of claim 2, wherein at least one setting of the respiratory therapy device is based at least in part on the first sensor data.

6. The method of claim 1, wherein the head-worn assembly includes i) a headband; ii) an eyepatch; iii) an eye mask; iv) a head covering; v) a nasal strip; vi) an ear bud; or vii) any combination of i-vi.

7. The method of claim 1, wherein the electronics module is i) removably couplable to a coupling of the head-worn assembly; or ii) removably positionable within a pocket of the head-worn assembly.

8. The method of claim 1, wherein the electronics module is detachable from a remainder of the head-worn assembly and couplable to the user interface when the head-worn assembly is in the second configuration.

9. The method of claim 1, wherein the electronics module is coupled to the user interface via a band when the head-worn assembly is in the second configuration.

10. The method of claim 2, wherein collecting the second sensor data includes transmitting the second sensor data to the respiratory therapy device via a wired connection or a wireless connection between the electronics module and the user interface.

11. The method of claim 2, wherein collecting the second sensor data includes powering the electronics module via an electrical connection between the electronics module and the respiratory therapy device.

12. (canceled)

13. The method of claim 1, further comprising collecting third sensor data from the one or more sensors while the user wears the head-worn assembly in the first configuration after the user ceases to wear the user interface during the second sleep session.

14. The method of claim 1, further comprising presenting a stimulus to the user via one or more output devices associated with the head-worn assembly, the stimulus including i) a visual stimulus; ii) a tactile stimulus; iii) an aural stimulus; iv) an electrical stimulus; v) an olfactory stimulus; or vi) a chemical stimulus; or vii) any combination of i-vi.

15-30. (canceled)

31. A system, comprising:
a head-worn assembly having an electronics module including one or more sensors, the head-worn assembly wearable in a first configuration to collect first sensor data associated with a user engaging in a first sleep session without receiving respiratory therapy; and
a user interface for supplying air to an airway of a user from a respiratory therapy device, wherein, when the head-worn assembly is in a second configuration, the head-worn assembly is reversibly coupled to the user interface to collect second sensor data associated with the user engaging in a second sleep session while receiving respiratory therapy via the user interface.

32. The system of claim 31, wherein the electronics module is i) removably couplable to a coupling of the head-worn assembly; or ii) removably positionable within a pocket of the head-worn assembly.

33. The system of claim 31, wherein the head-worn assembly includes i) a headband; ii) an eyepatch; iii) an eye mask; iv) a head covering; v) a nasal strip; vi) an ear bud; or vii) any combination of i-vi.

34. The system of claim 31, wherein the electronics module is detachable from a remainder of the head-worn assembly and couplable to the user interface when the head-worn assembly is in the second configuration.

35. (canceled)

36. The system of claim 31, wherein the user interface or the respiratory therapy device includes an antenna for wirelessly receiving the second sensor data from the electronics module when the head-worn assembly is worn in the second configuration.

37. The system of claim 31, wherein the user interface or the respiratory therapy device includes an electrical connector shaped to mate with an electrical connector of the head-worn assembly.

38. (canceled)

39. The system of claim 31, wherein the user interface includes a removable band to facilitate securing the user interface to the user, wherein when in the second configuration, at least a portion of the head-worn assembly replaces the removable band.

40. A head-worn assembly, comprising:
an electronics module including one or more sensors, and coupling means to couple to a user interface of a respiratory therapy system,
wherein the head-worn assembly is configured to be wearable in a first configuration to collect first sensor data associated with a user engaging in a first sleep session without receiving respiratory therapy; and
wherein the head-worn assembly is configured to be wearable in a second configuration and reversibly coupled to the user interface to collect second sensor data associated with the user engaging in a second sleep session while receiving respiratory therapy via the user interface.

41-48. (canceled)
